SEQUENCE LISTING (33 sequences)

SEQ ID NO: 1 - protein, 631 aa, Hepatitis C virus
APITAYAQQTRGLLGCIITGLTGRDKNQVEGEVQIVSTAAQTFLATCINGVCWTVYHGAGTRTIASSKGPVIQMYTNVDQDLVGWPAPQGARSLTPCTCGSSDLYLVTRHADVIPVRRRGDGRGSLLSPRPISYLKGSSGGPLLCPAGHAVGIFRAAVCTRGVAKAVDFIPVEGLETTMRSPVFSDNSSPPAVPQSYQVAHLHAPTGSGKSTKVPAAYAAQGYKVLVLNPSVAATLGFGAYMSKAHGIDPNIRTGVRTITTGSPITYSTYGKFLADGGCSGSAYDIIICDECHSTDATSILGIGTVLDQAETAGARLTVLATATPPGSVTVPHPNIEEVALSTTGEIPFYGKAIPLEAIKGGRHLIFCHSKKKCDELAAKLVALGVNAVAYYRGLDVSVIPASGDVVVVATDALMTGFTGDFDSVIDCNTCVTQTVDFSLDPTFTIETTTLPQDAVSRTQRRGRTGRGKPGIYRFVTPGERPSGMFDSSVLCECYDAGCAWYELTPAETTVRLRAYMNTPGLPVCQDHLEFWEGVFTGLTHIDAHFLSQTKQSGENLPYLVAYQATVCARAQAPPPSWDQMWKCLIRLKPTLHGPTPLLYRLGAVQNEITLTHPITKYIMTCMSADLEVVT

SEQ ID NO: 2 - protein, 591 aa, Hepatitis C virus
SMSYSWTGALITPCSAEEEKLPISPLSNSLLRHHNLVYSTSSRSASQRQRKVTFDRLQVLDDHYKTALKEVKERASKVKARMLTIEEACALVPPHSARSKFGYSAKDVRSLSSRAINQIRSVWEDLLEDATTPIPTTIMAKNEVFCVDPAKGGRKPARLIVYPDLGVRVCEKRALYDVIQKLSIETMGSAYGFQYSPQQRVERLLKMWTSKKTPLGFSYDTRCFDSTVTEQDIRVEEEIYQCCNLEPEARKVISSLTERLYCGGPMFNSKGAQCGYRRCRASGVLPTSFGNTITCYIKATAAAKAAGLRNPDFLVCGDDLVVVAESDGIDEDKAALRAFTEAMTRYSAPPGDVPQPTYDLELITSCSSNVSVARDNKGRRYYYLTRDATTPLARAAWETARHTPVNSWLGNIIMYAPTIWVRMVMMTHFFSILQSQEILDRPLDFEMYGATYSVTPLDLPAIIERLHGLSAFTLHSYSPVELNRVAGTLRKLGCPPLRAWRHRARAVRAKLIAQGGRAKICGLYLFNWAVRTKTNLTPLPTAGQLDLSSWFTVGVGGNDIYHSVSRARTRYLLLCLLLLTVGVGIFLLPAR

SEQ ID NO: 3 - protein, 626 aa, Human pegivirus
APVVIRRCGKGFLGVTKAALTGRDPDLHPGNVMVLGTATSRSMGTCLNGLLFTTFHGASSRTIATPVGALNPRWWSASDDVTVYPLPDGATSLTPCTCQAESCWVIRSDGALCHGLSKGDKVELDVAMEVSDFRGSSGSPVLCDEGHAVGMLVSVLHSGGRVTAARFTRPWTQVPTDAKTTTEPPPVPAKGVFKEAPLFMPTGAGKSTRVPLEYDNMGHKVLILNPSVATVRAMGPYMERLAGKHPSIYCGHDTTAFTRITDSPLTYSTYGRFLANPRQMLRGVSVVICDECHSHDSTVLLGIGRVRELARGCGVQLVLYATATPPGSPMTQHPSIIETKLDVGEIPFYGHGIPLERMRTGRHLVFCHSKAECERLAGQFSARGVNAIAYYRGKDSSIIKDGDLVVCATDALSTGYTGNFDSVTDCGLVVEEVVEVTLDPTITISLRTVPASAELSMQRRGRTGRGRSGRYYYAGVGKAPAGVVRSGPVWSAVEAGVTWYGMEPDLTANLLRLYDDCPYTAAVAADIGEAAVFFSGLAPLRMHPDVSWAKVRGVNWPLLVGVQRTMCRETLSPGPSDDPQWAGLKGPNPVPLLLRWGNDLPSKVAGHHIVDDLVRRLGVAEGYVRC

SEQ ID NO: 4 - protein, 357 aa, Human pegivirus
GCLVGNVLSFECDKCEARQETLASFSYIWSGVPLTRATPAKPPVVRPVGSLLVADTTKVYVTNPDNVGRRVDKVTFWRAPRVHDKFLVDSIERARRAAQACLSMGYTYEEAIRTVRPHAAMGWGSKVSVKDLATPAGKMAVHDRLQEILEGTPVPFTLTVKKEVFFKDRKEEKAPRLIVFPPLDFRIAEKLILGDPGRVAKAVLGGAYAFQYTPNQRVREMLKLWESKKTPCAICVDATCFDSSITEEDVALETELYALASDHPEWVRALGKYYASGTMVTPEGVPVGERYCRSSGVLTTSASNCLTCYIKVKAACERVGLKNVSLLIAGDDCLIICERPICDPSEALGRALASYGY

SEQ ID NO: 5 - DNA, 24 bp, synthetic construct
  source 1..24: mol_type = other DNA
gtgacctggtatggaatggaacct

SEQ ID NO: 6 - DNA, 24 bp, synthetic construct
  source 1..24: mol_type = other DNA
gtaacctggtatggaatggaacct

SEQ ID NO: 7 - DNA, 18 bp, synthetic construct
  source 1..18: mol_type = other DNA
cgacggctgcggtgtaag

SEQ ID NO: 8 - DNA, 20 bp, synthetic construct
  source 1..20: mol_type = other DNA
tgttggccctaccggtgtta

SEQ ID NO: 9 - DNA, 18 bp, synthetic construct
  source 1..18: mol_type = other DNA
ccgtacgtgggcgtcgtt

SEQ ID NO: 10 - DNA, 29 bp, synthetic construct
  source 1..29: mol_type = other DNA
agctaacctattgagactttacgacgact

SEQ ID NO: 11 - DNA, 29 bp, synthetic construct
  source 1..29: mol_type = other DNA
agctaacctactgagactttacgacgact

SEQ ID NO: 12 - DNA, 29 bp, synthetic construct
  source 1..29: mol_type = other DNA
agctaaccttctgagactttacgacgact

SEQ ID NO: 13 - DNA, 24 bp, synthetic construct
  source 1..24: mol_type = other DNA
ctcgtcgttaaaccgagcccgtca

SEQ ID NO: 14 - DNA, 24 bp, synthetic construct
  source 1..24: mol_type = other DNA
ctcgtcgttaaaccgagaccgaca

SEQ ID NO: 15 - DNA, 24 bp, synthetic construct
  source 1..24: mol_type = other DNA
ctcgtcgttaaaccgagaccgtca

SEQ ID NO: 16 - DNA, 20 bp, synthetic construct
  source 1..20: mol_type = other DNA
gcctgcctgatcctccaaat

SEQ ID NO: 17 - DNA, 21 bp, synthetic construct
  source 1..21: mol_type = other DNA
agggtagcggatgattcagcc

SEQ ID NO: 18 - DNA, 20 bp, synthetic construct
  source 1..20: mol_type = other DNA
tgtgtgctctcccaggtcta

SEQ ID NO: 19 - DNA, 20 bp, synthetic construct
  source 1..20: mol_type = other DNA
cagttctggatggtaactgg

SEQ ID NO: 20 - DNA, 20 bp, synthetic construct
  source 1..20: mol_type = other DNA
gacattccggaactcgtcaa

SEQ ID NO: 21 - DNA, 22 bp, synthetic construct
  source 1..22: mol_type = other DNA
cgtactccttcacctcaaactc

SEQ ID NO: 22 - DNA, 22 bp, synthetic construct
  source 1..22: mol_type = other DNA
ctggacaagagcagagatacac

SEQ ID NO: 23 - DNA, 18 bp, synthetic construct
  source 1..18: mol_type = other DNA
agcaacgatgcccatgat

SEQ ID NO: 24 - DNA, 22 bp, synthetic construct
  source 1..22: mol_type = other DNA
gttgtgcatccgaaagttcttc

SEQ ID NO: 25 - DNA, 22 bp, synthetic construct
  source 1..22: mol_type = other DNA
ggttcagctcttcctcctttag

SEQ ID NO: 26 - DNA, 22 bp, synthetic construct
  source 1..22: mol_type = other DNA
aggtacttctgctccaggatag

SEQ ID NO: 27 - DNA, 22 bp, synthetic construct
  source 1..22: mol_type = other DNA
cgacctgagcctcatgaataaa

SEQ ID NO: 28 - DNA, 20 bp, synthetic construct
  source 1..20: mol_type = other DNA
ggcgtcatgcatctgttcta

SEQ ID NO: 29 - DNA, 22 bp, synthetic construct
  source 1..22: mol_type = other DNA
cacgggcacatataccaagtag

SEQ ID NO: 30 - DNA, 19 bp, synthetic construct
  source 1..19: mol_type = other DNA
gctggaacaggtgcctaaa

SEQ ID NO: 31 - DNA, 20 bp, synthetic construct
  source 1..20: mol_type = other DNA
ccgtccagtgtcaggttatc

SEQ ID NO: 32 - DNA, 23 bp, synthetic construct
  source 1..23: mol_type = other DNA
tgacttcaacagcgacacccact

SEQ ID NO: 33 - DNA, 24 bp, synthetic construct
  source 1..24: mol_type = other DNA
accaccctgttgctgtagccaaat

(19) United States
(12) Patent Application Publication
(10) Pub. No.: US 2024/0287575 A1
(43) Pub. Date: Aug. 29, 2024

(54) Role of Human Pegivirus (HPGV) in Parkinson's Disease: Implications for Diagnosis and Treatment

(71) Applicants: Northwestern University, Evanston, IL (US); Rush University Medical Center, Chicago, IL (US)

(72) Inventors: Barbara Alice Hanson, Evanston, IL (US); Igor Koralnik, Chicago, IL (US)

(21) Appl. No.: 18/587,523

(22) Filed: Feb. 26, 2024

Related U.S. Application Data
(60) Provisional application No. 63/486,951, filed on Feb. 24, 2023.

(51) Int. Cl. C12Q 1/6804; A61K 31/381; A61K 31/403; A61K 31/501; C12N 15/10; C12Q 1/70

(52) U.S. Cl. CPC C12Q 1/6804; A61K 31/381; A61K 31/403; A61K 31/501; C12N 15/1096; C12Q 1/70; C12Q 2600/118; C12Q 2600/158

(57) ABSTRACT

The present invention provides methods for diagnosing and treating human pegivirus infection and Parkinson's disease.

### CROSS-REFERENCE TO RELATED APPLICATIONS

This application claims priority to U.S. Provisional Application No. 63/486,951, filed on Feb. 24, 2023, the contents of which are incorporated by reference in their entireties.

### STATEMENT REGARDING THE SEQUENCE LISTING

This application includes a sequence listing in XML format titled “702581.02481_ST26.xml”, which is 6,691 bytes in size and was created on Feb. 1, 2024. The sequence listing is electronically submitted with this application via Patent Center and is incorporated herein by reference in its entirety.

### BACKGROUND

Parkinson's disease (PD) is a progressive neurodegenerative movement disorder of unknown etiology that results from the loss of dopaminergic neurons. PD affects 1 million people in the US. There is currently no cure for this disease and only limited symptomatic treatments are available. Genetic associations for PD account for only a small proportion of PD cases, and it is widely believed that PD risk variants interact with environmental factors to cause disease onset. Viral infections represent a significant environmental risk factor for PD. However, while many researchers have looked for a viral cause or trigger of PD, they have all failed to obtain conclusive evidence. Accordingly, there is a need in the art for a better understanding of the risk factors that lead to the development of PD.

### SUMMARY

In a first aspect, the present invention provides methods of diagnosis. The methods comprise detecting a biomarker associated with human pegivirus (HPgV) in a sample from a subject. In some embodiments, the methods are used to diagnose an HPgV infection in the subject. In other embodiments, the methods are used to diagnose Parkinson's disease (PD) in the subject.

In a second aspect, the present invention provides methods of treatment. The methods comprise administering an anti-hepatitis C virus (HCV) drug to a subject. In some embodiments, the methods are used to treat an HPgV infection in the subject. In other embodiments, the methods are used to treat PD in the subject.

### BRIEF DESCRIPTION OF THE DRAWINGS

The patent or patent application file contains at least one drawing in color. Copies of this patent or patent application publication with color drawings will be provided by the Office upon request and payment of the necessary fec.

FIG. 1 is a heatmap showing results of a ViroFind analysis of samples from Parkinson's disease (PD) patients (left) and non-PD controls (right). The color saturation of the boxes are weighted on a log 2 scale of the number of reads mapped to the viral species indicated along the y-axis.

FIGS. 2A-2B show immunofluorescence staining for HPgV in a slice of the posterior putamen (PPUT) from a PD patient. Images were taken at 40×. FIG. 2A shows co-localization of the astrocyte marker glial fibrillary acidic protein (GFAP) with the HPgV-specific protein envelope protein 2 (E2). No E2 signal was detected in GFAP positive cells in control PPUT slices. FIG. 2B shows nuclei stained with DAPI in an uninfected astrocyte.

FIG. 3 is a coverage analysis of contigs and loose reads obtained from cerebrospinal fluid (CSF) of PD patients, which shows full genome-length coverage for ⅔ PD patients and 80% coverage for ⅓ PD patients against a reference sequence (KP710598.1).

FIGS. 4A-4C show phenotypic changes in astrocytes following infection with HPgV. FIG. 4A shows a significant decrease in mitochondrial DNA (mtDNA) copy number in both normal human astrocytes (NHA) and U87 mg glioblastoma cells infected with HPgV as compared to mock infected cells. No significant change to mtDNA copy number observed in cytomegalovirus (CMV)-infected cells. FIG. 4B shows changes in expression of the PD astrocyte associated genes glial fibrillary acidic protein (GFAP), lipocalin 2 (LCN2), α-synuclein (SNCA), and inositol 1,4,5-triphosphate receptor 2 (ITPR2) in HPgV-infected NHA. FIG. 4C shows changes in expression of the A1 phenotype genes complement 3 (C3), MHC class 1 (HLA-C), guanylate binding protein (Gbp2), and Amigo and the A2 phenotype genes sphingosine kinase 1 (Sphk1) and CD14 in HPgV-infected NHA. * p<0.05; ** p<0.005; ***p<0.0005.

FIGS. 5A-5C show PD-associated changes in HPgV-positive (HPgV+) NHA following cytokine exposure. FIG. 5A shows the mtDNA concentration in HPgV+ NHA (left) and HPgV+ U87 mg (right) following exposure to the cytokines IL1β, IFNγ, and TNFα (light gray bars) as compared to the mtDNA concentration in uninfected cells following cytokine exposure (dark gray bars). FIG. 5B shows expression levels of PD-associated astrocyte genes (GFAP, LCN2, and SNCA) in HPgV+ NHA following cytokine exposure as compared to in uninfected NHA following cytokine exposure. FIG. 5C shows expression of A1 phenotype (C3, HLA-C, Gbp2, and Amigo) and A2 phenotype (Sphk1 and CD14) astrocyte genes following exposure to cytokines in HPgV+ NHA. * p<0.05; ** p<0.005; *** p<0.0005.

FIGS. 6A-6B show the viability of co-cultured SHSY5Y and NHA cells based on a dye exclusion assay. FIG. 6A shows the viability of SHSY5Y cells following 7 days of co-culture with uninfected or HPgV-infected NHA. FIG. 6B shows the viability of uninfected (HPgV−) and HPgV-infected (HPgV+) NHA. * p<0.05.

FIG. 7 shows immunostaining for α-synuclein (SNCA) in U87 mg/SHSY5Y co-culture. Mixed cultures of HPgV-infected U87 mg and SHSY5Y cells (lanes 1-2) show increased accumulation of SNCA protein (middle) as compared to mixed culture of uninfected U87 mg and SHSY5Y (lanes 3-4) at day 7. Uninfected U87 mg alone (lane 5) and HPgV-infected U87 mg alone (lane 6) do not show accumulation of SNCA. Levels of the HPgV protein NS5A are used as an indicator of productive infection (lanes 1-2). Tubulin is used as loading control.

FIG. 8 is a graphical schematic of the computational drug-repurposing pipeline used in Example 5.

FIGS. 9A-9F show GOLD docking positions of the best drugs that target the HPgV proteins NS3 and NS5B. FIG. 9A shows a cartoon structure of the HPgV NS3 protein with one chain colored green and the other cyan and with the drug tegobuvir docked in the active site. Tegobuvir is shown as sticks with carbons colored pink and a mesh surface. FIG. 9B shows a zoomed-in image of tegobuvir docked in the HPgV NS3 active site (colored blue). HPgV NS3 is shown as a green surface at 50% transparency. FIG. 9C shows a stick representation of the intermolecular interactions of tegobuvir and HPgV NS3. FIG. 9D shows a cartoon structure of the HPgV NS5B protein with the drug lomibuvir docked in the active site. Lomibuvir is shown as sticks with carbons colored pink and a mesh surface. FIG. 9E shows a zoomed-in image of lomibuvir docked in the HPgV NS5B active site (colored blue). HPgV NS5B is shown as an orange surface at 50% transparency. FIG. 9F shows a stick representation of the intermolecular interactions of lomibuvir and HPgV NS5B.

FIGS. 10A-10B show structural alignments of HPgV and hepatitis C virus (HCV) proteins. FIG. 10A shows an alignment of the NS3 proteins from HPgV (blue) and HCV (red). FIG. 10B shows an alignment of the NS5B proteins from HPgV (blue) and HCV (red).

FIG. 11 shows the predicted active site residues of HCV NS3, HPgV NS3, and HCV NS5B based on an I-TASSER analysis.

FIGS. 12A-12B are tables that describe the anti-HCV drugs that were analyzed in Example 5. The drugs included in FIG. 12A are FDA approved, and the drugs included in FIG. 12B are in clinical trials.

FIGS. 13A-13B are tables that characterize the bioavailability, drug-likeness, and blood-brain barrier (BBB) permeability (log BB) of anti-HCV drugs. FIG. 13A shows the results generated for the top five NS3-targeting drugs, and FIG. 13B shows the results generated for the top 5 NS5B-targeting drugs.

### DETAILED DESCRIPTION

The present invention provides methods for diagnosing and treating human pegivirus (HPgV) infection and Parkinson's disease (PD).

To identify viruses that may play a role in the development of PD, the inventors performed unbiased, whole virome sequencing on frozen postmortem brain tissue and cerebrospinal fluid (CSF) samples from PD patients. They detected the presence of HPgV RNA and protein in samples from PD patients but not in samples from patients with other neurodegenerative diseases or with no known brain diseases (Example 1). They further showed that astrocytes are the primary cell type infected by HPgV in the PD brain (Example 1), that HPgV-infected astrocytes exhibit phenotypes observed in PD (Example 2), and that HPgV-infected astrocytes negatively affect neighboring neurons (Example 3). Thus, their work implicates HPgV as a potential cause or trigger of PD.

Because HPgV is considered non-pathogenic, treatments for this virus are not available. However, HPgV is closely related to hepatitis C virus (HCV), for which treatments are available. Thus, if HPgV is indeed a causal or triggering factor for PD, repurposed or modified anti-HCV drugs could potentially be used to treat or prevent PD. By performing a structural analysis of HCV and HPgV proteins, the inventors have identified several anti-HCV drugs that are promising candidates for use in treating PD (Example 5).

### Methods of Diagnosis:

In a first aspect, the present invention provides methods of diagnosis. The methods may be used to diagnose HPgV infection or PD in a subject. In both cases, the methods comprise detecting a biomarker associated with HPgV in a sample from the subject.

In some embodiments, the methods are used to diagnose an HPgV infection in a subject. Human pegivirus (HPgV), also referred to as GB virus C (GBVC) and Hepatitis G Virus (HGV), is a member of the Flavivirus family of RNA viruses. It has a positive-sense RNA genome of about 9.3 kb that is translated into a single polyprotein that is then cleaved into mature viral proteins. HPgV is a blood-borne virus that is commonly transmitted via contaminated blood, intravenous drug use, sexual contact, and mother-to-child transmission. However, HPgV is not known to be pathogenic in humans, except for in very rare cases. Thus, as used herein, the term “HPgV infection” refers merely to the presence of HPgV in a subject's cells.

As is described in the Examples, the inventors discovered that HPgV RNA and protein is present in samples from PD patients but not from healthy controls. Thus, in some embodiments, the HPgV infection is indicative of PD or an increased risk for developing PD.

In other embodiments, the methods are used to diagnose PD in a subject. Parkinson's disease (PD) is a neurodegenerative disease caused by loss of dopamine-producing neurons in the brain. Common symptoms of PD include shaking (e.g., tremors in the hands, arms, legs, jaw, or head), muscle stiffness, slowness of movement, and impaired balance and coordination. Additional symptoms may include depression; difficulty swallowing, chewing, and speaking; urinary problems or constipation; and skin problems. Symptoms usually begin gradually and worsen over time, and as the disease progresses, people may have difficulty walking and talking. Current treatments for PD are limited to levodopa and dopamine agonists, which have a limited a half-life and cause numerous side effects.

The term “biomarker” refers to a biological molecule that is associated with or indicative of a particular disease, condition, or cell state. Suitable biomarkers include, for example, nucleic acids, proteins, lipids, sugar moieties, hormones, cytokines, and the like. Biomarkers can be used as part of a predictive, prognostic, or diagnostic process. For example, biomarkers may be used to predict the likelihood that a particular subject will respond well to a particular therapeutic. In some cases, the mere presence (or absence) of a biomarker in a biological sample is indicative of a particular condition, whereas in other cases the biomarker is only indicative of a condition when it is present at a particular level or in a specific location within a biological sample. The biomarkers used in the methods of the present invention are associated with HPgV, meaning that they are indicative of the presence of HPgV (i.e., HPgV infection).

As is described in the Examples, the inventors first detected HPgV in samples from PD patients using RNA sequencing. Thus, in some embodiments, the biomarker is RNA that is associated with HPgV. The biomarker may be a positive-sense RNA found in the genome of HPgV or may be negative-sense RNA produced therefrom during viral replication. RNA biomarkers may be detected using any suitable method of RNA detection including, without limitation, reverse transcription polymerase chain reaction (RT-PCR), RNase protection assays, Northern blotting, in situ hybridization, microarray analysis, and RNA sequencing. To facilitate detection, the RNA may first be isolated from cells using an RNA extraction technique, such as guanidinium thiocyanate-phenol-chloroform extraction (e.g., using TRIzol), trichloroacetic acid/acetone precipitation followed by phenol extraction, or using a commercially available column-based system (e.g., RNeasy RNA Preparation Kit from Qiagen). Such techniques are well known in the art.

In other embodiments, the biomarker is an anti-HPgV antibody. The term “antibody” refers to a protein that comprises at least one antigen-binding domain from an immunoglobulin molecule. An anti-HPgV antibody is an antibody that specifically binds to an HPgV antigen. Antibody biomarkers can be detected using any suitable method of antibody detection including, without limitation, immunoprecipitation, immunocytochemistry, immunoblotting, and enzyme-linked immunosorbent assays (ELISA). A variety of ELISA kits for antibody detection are commercially available.

In other embodiments, the biomarker is an HPgV-specific CD8⁺ T cell response. A “CD8⁺ T cell,” which is also known as a cytotoxic T cell, is a type of cell that can kill damaged, infected, cancerous, or otherwise targeted cells. CD8⁺ T cells express T-cell receptors (TCRs) that recognize a specific antigen. If a TCR of a CD8⁺ T cell recognizes an antigen presented by a class I MHC molecule of a somatic cell, the CD8⁺ T cell will destroy that cell. As used herein, an “HPgV-specific CD8⁺ T cell” is a CD8⁺ T cell that recognizes an antigen from HPgV. After recognizing a cell, CD8⁺ T cells can produce cytokines (e.g., IFN-γ, TNF-α, IL-17), chemokines (e.g., IP-10, lymphotactin), and cytotoxins (e.g., perforin, granzymes, granulysin) and can trigger cytolysis of target cells. Thus, a CD8⁺ T cell response can be detected using any assay that measures one of these outcomes. Such assays are referred to herein as “CD8⁺ T cell cytotoxicity assays.” Examples of CD8⁺ T cell cytotoxicity assays include, without limitation, assays that measure IFNγ production (e.g., via ELISPOT), assays that measure intracellular cytokines or cytotoxic granules (e.g., via flow cytometry), and direct cytotoxicity assays (live/dead target cell assays).

As used herein, the term “sample” refers to a sample taken from the subject. Suitable samples include tissue samples (e.g., brain tissue) and fluid samples (e.g., saliva, blood, serum, plasma, urine, stool, cerebrospinal fluid). In the Examples, the inventors demonstrate that HPgV RNA can be detected in cerebrospinal fluid (CSF) samples from patients with PD. Thus, in some embodiments, the sample used in the method is a CSF sample. In other embodiments, the sample is a serum or blood sample.

The “subject” to which the methods are applied may be a mammal or a non-mammalian animal, such as a bird. Suitable mammals include, but are not limited to, humans, cows, horses, sheep, pigs, goats, rabbits, dogs, cats, bats, mice, and rats. In certain embodiments, the methods may be performed on lab animals (e.g., mice and rats) for research purposes. In other embodiments, the methods are used to treat commercially important farm animals (e.g., cows, horses, pigs, rabbits, goats, sheep, and chickens) or companion animals (e.g., cats and dogs). In a preferred embodiment, the subject is a human.

### Methods of Treatment:

In a second aspect, the present invention provides methods of treatment. The methods may be used to treat an HPgV infection or PD in a subject. In both cases, the methods comprise administering an anti-hepatitis C virus (HCV) drug to the subject.

As used herein, “treating” or “treatment” describes something that is done to a subject to combat a health problem (i.e., a disease, condition, or disorder). Treating may involve controlling the health problem, lessening the symptoms or complications of the health problem, and/or eliminating the health problem.

As used herein, the term “administering” refers to the introduction of a substance into a subject's body. Methods of administration are well known in the art and include, but are not limited to, oral administration, transdermal administration, administration by inhalation, nasal administration, topical administration, intravaginal administration, ophthalmic administration, intraoral administration, intracerebral administration, rectal administration, sublingual administration, buccal administration, and parenteral administration, including injectable such as intravenous administration, intra-arterial administration, intramuscular administration, intradermal administration, intrathecal administration, and subcutaneous administration. Administration can be continuous or intermittent.

As used herein, an “anti-hepatitis C virus (HCV) drug” is an antiviral medication that has been shown to treat HCV. Like HPgV, HCV is a positive-sense single-stranded RNA virus of the family Flaviviridae. HCV is the cause of hepatitis C and some cancers, such as liver cancer and lymphomas. It is estimated that 12-37% of the HCV infected population is also infected with HPgV-1. In Example 5, the inventors identified two HPgV proteins, i.e., NS3 and NS5B, as promising HPgV drug targets. Thus, in some embodiments, the anti-HCV drug is an NS3-targeting drug or an NS5B-targeting drug.

The HPgV NS3 protein (SEQ ID NO: 3) has helicase and protease activities. As used herein, an “NS3-targeting drug” is a drug that binds to NS3 and inhibits its helicase activity and/or protease activity. Examples of suitable NS3-targeting drugs include, without limitation, boceprevir, glecaprevir, grazoprevir, paritaprevir, simeprevir, telaprevir, asunaprevir, danoprevir, narlaprevir, and tegobuvir.

The HPgV NS5B protein (SEQ ID NO: 4) encodes an RNA-dependent RNA polymerase and is crucial for viral replication. As used herein, an “NS5B-targeting drug” is a drug that binds to NS5B and inhibits viral replication. Examples of suitable NS5B-targeting drugs include, without limitation, dasabuvir, sofosbuvir, mercitibane, lomibuvir, 2′-C′-methylguanosine, PSI-7976, Ro-9187, 4′-azidocytidine, and VCH-916.

Based on their structural analysis of HCV and HPgV proteins, the inventors identified the anti-HCV drugs tegobuvir, boceprevir, and lomibuvir as the most promising candidates for use for treating PD (see Example 5). Thus, in some embodiments, the anti-HCV drug is tegobuvir, boceprevir, or lomibuvir.

In some embodiments, the anti-HCV drug is modified. For example, the anti-HCV drug may be modified to make it more effective for treating an HPgV infection or PD, to have improved pharmacological properties, to have increased bioavailability in the central nervous system, or to have reduced side effects. In some embodiments, the anti-HCV drug is modified to be a prodrug (i.e., an inactive form) that must be converted to pharmacologically active form in vivo. For example, the prodrug may be formulated such that it is activated after it has entered an infected cell. In some embodiments, the anti-HCV drug is modified to allow it to cross the blood brain barrier (BBB), i.e., the highly selective semipermeable border of endothelial cells that prevents solutes in the circulating blood from crossing into the extracellular fluid of the central nervous system.

In preferred embodiments, a therapeutically effective amount of an anti-HCV drug is administered to the subject. The term “therapeutically effective amount” refers to an amount that is sufficient to effect a beneficial or desirable clinical result. The result can be reducing, preventing, or delaying the onset of one or more symptoms of a disease or condition. For any active agent, a therapeutically effective amount can be estimated initially in cell culture assays or in animal models (e.g., mice, rabbits, dogs, pigs). An animal model may also be used to determine a desirable concentration range and route of administration.

In some embodiments, the methods further comprise detecting a biomarker associated with HPgV in a sample from the subject prior to administering an anti-HCV drug to the subject. This additional step is performed to determine whether the subject has an HPgV infection, as this may suggest that the anti-HCV drug is likely to be effective. In these embodiments, the sample may be a CSF sample, a serum sample, or a blood sample. Further, in these embodiments, the biomarker may be an RNA associated with HPgV, an anti-HPgV antibody, or an HPgV-specific CD8⁺ T cell response. Suitable methods for detecting these biomarkers are described above.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

The following examples are meant only to be illustrative and are not meant as limitations on the scope of the invention or of the appended claims.

### EXAMPLES

### Example 1: Human Pegivirus is Present in the Brains of Parkinson's Disease Patients

To identify viruses that may play a role in the development of Parkinson's disease (PD), the inventors utilized an in-house assay called ViroFind to perform whole virome sequencing on samples from PD patients. They detected the presence of human pegivirus (HPgV) RNA in 50% (5/10) of the postmortem brain tissue samples from the PD patients but in 0% (0/12) of non-PD control brains, demonstrating that the prevalence of HPgV is significantly increased in the brains of PD patients. Additionally, they determined that astrocytes are the primary cell type infected by HPgV in the PD brain and that PD patients have a high frequency of HPgV RNA in the cerebrospinal fluid (CSF) but not in the periphery. Collectively, these results suggest that HPgV infection may be involved in the etiology or pathogenesis of PD.

Note: The data and descriptions presented in Examples 1-4 are part of a published dissertation (Hanson, BA (2022). Human Pegivirus in Parkinson's disease. (Thesis No. 28773352). [Doctoral dissertation, Rush University]. ProQuest), which is hereby incorporated by reference in its entirety.

### Results:

### ViroFind Identifies HPgV in Parkinson's Disease Brains

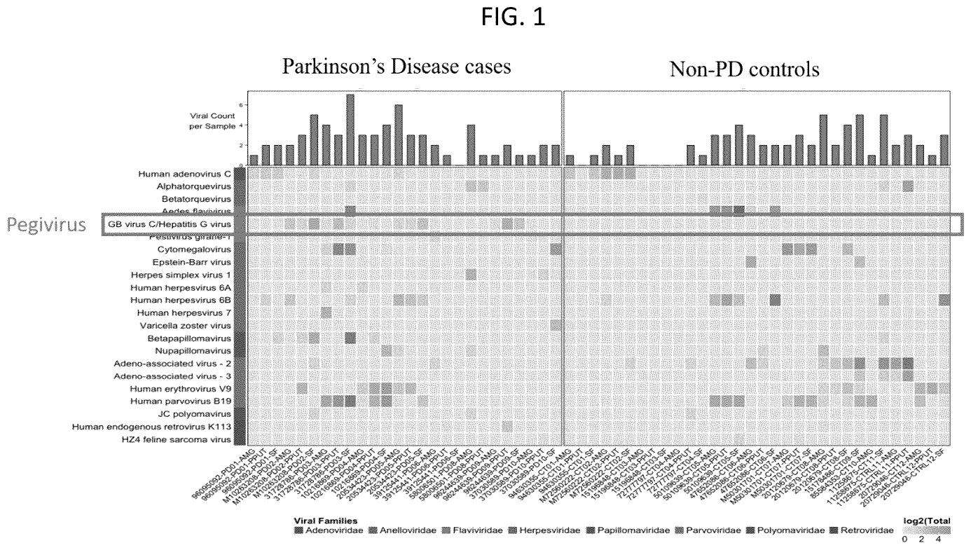

To assess viral involvement in PD, a ViroFind analysis was performed on postmortem brain samples from 10 PD patients and 12 demographically matched controls. Three separate brain regions relevant to PD pathogenesis, i.e., the superior frontal cortex (SFC), posterior putamen (PPUT), and amygdala (AMG) were analyzed for each subject. Significantly, HPgV sequences were identified in 50% (5/10) of the PPUT samples from the PD patients, and HPgV sequences were identified in all three of the analyzed brain regions for 40% (4/10) of the PD patients (FIG. 1). No samples from PD control patients (0/12) contained HPgV sequences (FIG. 1), nor has HPgV been observed in brain tissue samples from patients with other neurological diseases that were previously tested using ViroFind (n=96 samples: 8 multiple systems atrophy, 8 progressive supranuclear palsy, 30 multiple sclerosis, 24 amyotrophic lateral sclerosis, 20 Alzheimer's disease, and 6 progressive multifocal leukoencephalopathy).

qPCR Confirmation of HPgV in PD Brains

Extracted brain tissue RNA from the pilot study patients was screened with an in-house quantitative PCR (qPCR) assay targeting the NS3 and UTR regions of the HPgV genome. Samples were considered qualitatively positive for HPgV when both NS3 and UTR were detected with cycle threshold values of less than 38 cycles for at least one fluorophore in duplicate wells. No samples tested positive for HPgV by qPCR that did not test positive via ViroFind analysis. qPCR identified HPgV in ¾ of the HPgV-positive AMG samples, ¾ of the HPgV-positive SFC samples, and ⅘ of the HPgV-positive PPUT samples that were identified by ViroFind (Table 1).

TABLE 1
HPgV-positive samples identified by ViroFind and qPCR

| | | | Posterior | | Superior | | Total | |
|---|---|---|---|---|---|---|---|---|
| | Amygdala | | Putamen | | Frontal Cortex | | Regions | |
| PID* | ViroFind | qPCR | ViroFind | qPCR | ViroFind | qPCR | ViroFind | qPCR |
| PD01 | − | − | − | − | − | − | 0 | 0 |
| PD02 | + | + | + | − | + | + | 3 | 2 |
| PD03 | + | − | + | + | + | − | 3 | 1 |
| PD04 | − | − | + | + | − | − | 1 | 1 |
| PD05 | + | + | + | + | + | + | 3 | 3 |
| PD06 | − | − | − | − | − | − | 0 | 0 |
| PD07 | − | − | − | − | − | − | 0 | 0 |
| PD08 | − | − | − | − | − | − | 0 | 0 |
| PD09 | + | + | + | + | + | + | 3 | 3 |
| PD10 | | − | − | − | − | − | 0 | 0 |
| | 4/10 | 4/10 | 5/10 | 4/10 | 4/10 | 3/10 | 13/30 | 10/30 |
| CT01 | − | − | − | − | − | − | 0 | 0 |
| CT02 | − | − | − | − | − | − | 0 | 0 |
| CT03 | − | − | − | − | − | − | 0 | 0 |
| CT04 | − | − | − | − | − | − | 0 | 0 |
| CT05 | − | − | − | − | − | − | 0 | 0 |
| CT06 | − | − | − | − | − | − | 0 | 0 |
| CT07 | − | − | − | − | − | − | 0 | 0 |
| CT08 | − | − | − | − | − | − | 0 | 0 |
| CT09 | − | − | − | − | − | − | 0 | 0 |
| CT10 | − | − | − | − | − | − | 0 | 0 |
| CT11 | − | − | − | − | − | − | 0 | 0 |
| CT12 | − | − | − | − | − | − | 0 | 0 |
| | 0/12 | 0/12 | 0/12 | 0/12 | 0/12 | 0/12 | 0/36 | 0/36 |

*PID—Patient ID number

### Immunofluorescence Detection of HPgV in PD Brains

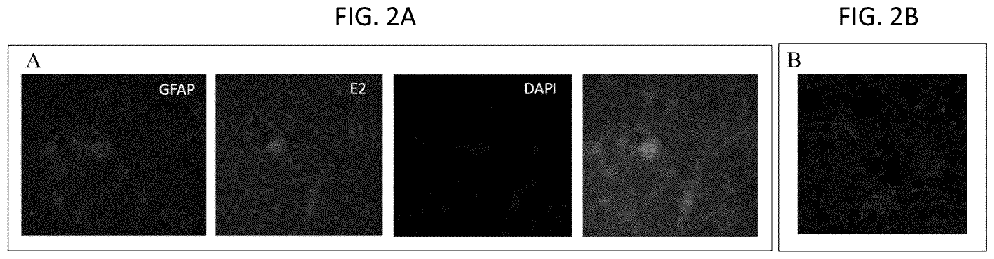

To determine which cells were infected with HPgV in the PD patient group, immunofluorescence staining was performed on contralateral, formalin-fixed, paraffin-embedded tissue slices. Three central nervous system (CNS) cell markers were used to differentiate between neurons (NeuN, #834501, Biolegend, 1:100), microglia (Iba1; #ab178846; Abcam; 1:750), and astrocytes (glial fibrillary acidic protein (GFAP); #Z0334; Dako; 1:500) alongside the lymphocyte marker CD3 (#A0452, Dako, 1:250). HPgV-specific antibodies against non-structural protein 5A (NS5A; #NB100-93584; Novus Biotechnologies; 1:100) and envelope protein 2 (E2; MA5-18088; Invitrogen; 1:100) were utilized, depending on species compatibility. HPgV-positive cells showed co-staining with GFAP, indicating that the HPgV-positive cells are astrocytes (FIGS. 2A-2B).

### HPgV in CSF of PD Patients

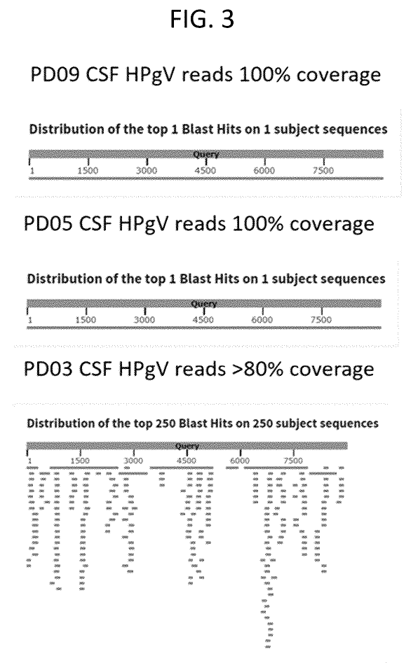

Cerebrospinal fluid (CSF) samples were also tested via ViroFind analysis, and samples from three of the PD patients were found to contain HPgV RNA. All three of these PD patients also tested positive for HPgV in all three of the tested brain regions. Full HPgV genome length sequences were obtained for ⅔ patients (FIG. 3). qPCR analysis was then performed as previously described, and all three CSF samples that tested positive for HPgV by ViroFind also tested positive by qPCR. No samples that tested negative for HPgV by ViroFind tested positive by qPCR (Table 2).

### Viremia and Seroprevalence of HPgV in PD Patients

Matched plasma samples were tested for HPgV RNA via ViroFind and qPCR analysis. HPgV RNA was not detected in any of the plasma samples from the PD patients or controls. Next, serology for HPgV-specific IgG was used to test whether any of the patients had evidence of a previous HPgV infection that was subsequently cleared. Two out of 11 (18.2%) of the control samples had positive IgG titers against HPgV envelope protein, while none (0/10; 0%) of the PD cases showed evidence of a prior infection (Table 2).

TABLE 2
Detection of HPgV RNA and HPgV-specific IgG in fluid samples

| | | |
|---|---|---|
| | | Plasma |
| CSF | Plasma | IgG |

| PID* | ViroFind | qPCR | ViroFind | qPCR | ELISA |
|---|---|---|---|---|---|
| PD01 | − | − | − | − | − |
| PD02 | − | − | − | − | − |
| PD03 | + | + | − | − | − |
| PD04 | − | − | − | − | − |
| PD05 | + | + | − | − | − |
| PD06 | − | − | − | − | − |
| PD07 | − | − | − | − | − |
| PD08 | − | − | − | − | − |
| PD09 | + | + | − | − | − |
| PD10 | − | − | − | − | − |
| | 3/10 | 3/10 | 0/10 | 0/10 | 0/10 |
| CT01 | − | − | − | − | − |
| CT02 | − | − | − | − | − |
| CT03 | − | − | − | − | + |
| CT04 | − | − | − | − | − |
| CT05 | − | − | − | − | − |
| CT06 | − | − | − | − | − |
| CT07 | − | − | − | − | − |
| CT08 | − | − | NA | NA | NA |
| CT09 | − | − | − | − | − |
| CT10 | − | − | − | − | + |
| CT11 | − | − | − | − | − |
| CT12 | − | − | − | − | − |
| | 0/12 | 0/12 | 0/11 | 0/11 | 2/11 |

*PID—Patient ID number

### Example 2: Human Pegivirus-Infected Astrocytes Exhibit Phenotypes that are Consistent with Parkinson's Disease

Current evidence suggests that glial cells play a significant role in PD pathogenesis. In PD, microglia become hyper-inflammatory and lose their ability to phagocytose α-synuclein (SNCA), leading to its accumulation and the formation of aggregates called Lewy Bodies. Astrocytes respond to these inflammatory stimuli by surrounding the area and producing neurotoxic factors, which leads to neuronal damage.

In the following example, the inventors demonstrate that HPgV-infected astrocytes exhibit decreased mitochondrial quality, adopt a neurotoxic A1 phenotype, and are hyper-responsive to inflammatory stimuli as compared to uninfected astrocytes. These findings suggest that HPgV infection promotes molecular changes in astrocytes that are consistent with the changes seen in PD.

### Results:

### Changes in Mitochondrial DNA Copy Number

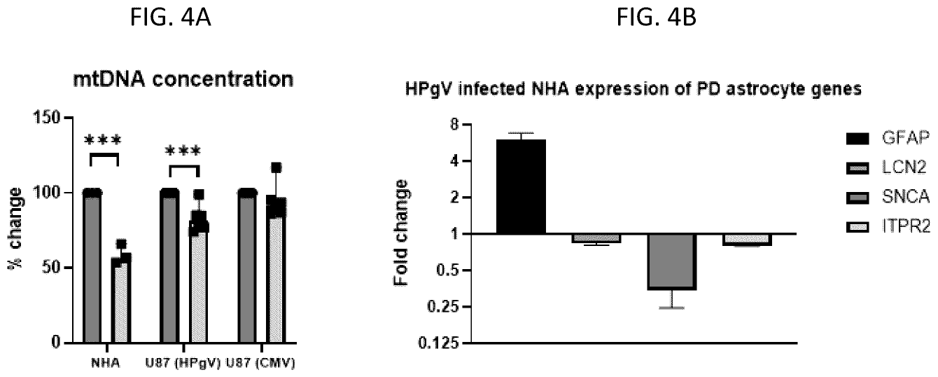

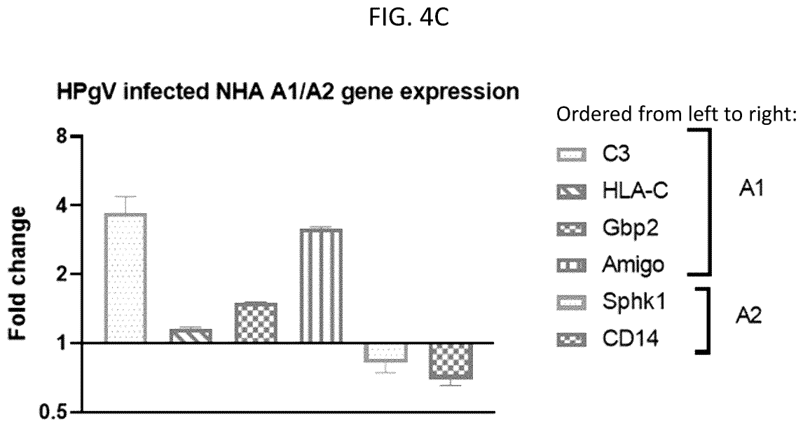

It has been shown that PD patients have reduced mitochondrial DNA (mtDNA) copy numbers in both affected brain regions and in peripheral blood mononuclear cells (PBMCs). Moreover, induced pluripotent stem cell (iPSC)-derived PD astrocytes have reduced mtDNA copy numbers as compared to isogenic controls.¹⁻³ Mitochondrial dysfunction is central to the etiology of PD. Thus, the ability of HPgV infection to induce a similar reduction of mtDNA copy number in permissive cells was assessed. A qPCR analysis was performed to quantify copies of a mitochondrially encoded gene (CYTB) as compared to a nuclear encoded gene (APP). Both normal human astrocytes (NHA) (p=0.0004) and U87 mg glioblastoma cells (p=0.0009) exhibited significantly reduced mtDNA copy numbers following HPgV infection as compared to mock infected cells, while human cytomegalovirus (HCMV)-infected U87 mg showed a non-significant reduction of mtDNA copy number (p=0.29) (FIG. 4A).

### Changes in Expression of PD Astrocyte Associated Genes

In addition to decreased mtDNA copy number, iPSC-derived PD astrocytes were shown to have intrinsic upregulation of inositol 1,4,5-triphosphate receptor type 2 (ITPR2) and inducible upregulation of lipocalin 2 (LCN2) and GFAP following exposure to inflammatory conditions. SNCA expression was also shown to be increased in iPSC-derived PD astrocytes, but required 4 months post differentiation to be consistently observed.¹ Therefore, the ability of HPgV infection to recapitulate these findings was tested by determining the change in expression of HPgV infection-associated genes in NHA and U87 mg (FIG. 4B). GFAP expression was increased 5-fold following HPgV infection in NHA, whereas expression of LCN2 and ITPR2 were unchanged, and SNCA expression was reduced by 2-fold. Unlike NHA, U87 mg showed a 4-fold induction of LCN2, and showed no changes in SNCA expression following HPgV infection as compared to uninfected U87 mg.

### Astrocytic A1 Skewing

Activated astrocytes exhibit two phenotypes: A1 astrocytes are neurotoxic and contribute to neurodegenerative diseases, whereas A2 astrocytes are neuroprotective. The observation of increased expression of GFAP is consistent with astrocytic A1 skewing (i.e., the process in which astrocytes adapt the A1 phenotype). Reactive astrocytes have been implicated in PD pathology based on data from both murine models and postmortem human studies.⁴ Additionally, agonist blockade of A1 astrocyte conversion has been shown to reduce PD pathology in murine models.⁵ Thus, the ability of HPgV infection to induce A1 skewing in NHA was tested via qPCR analysis of a panel of A1 genes and A2 genes (FIG. 4C). A >2-fold induction of the A1 astrocyte genes C3 and Amigo and a smaller increase in the A1 astrocyte genes Gbp2 and HLA-C was observed, whereas the A2 astrocyte genes Sphk1 and CD14 exhibited downregulation.

### Synergistic Effects of Exposure to Inflammatory Cytokines

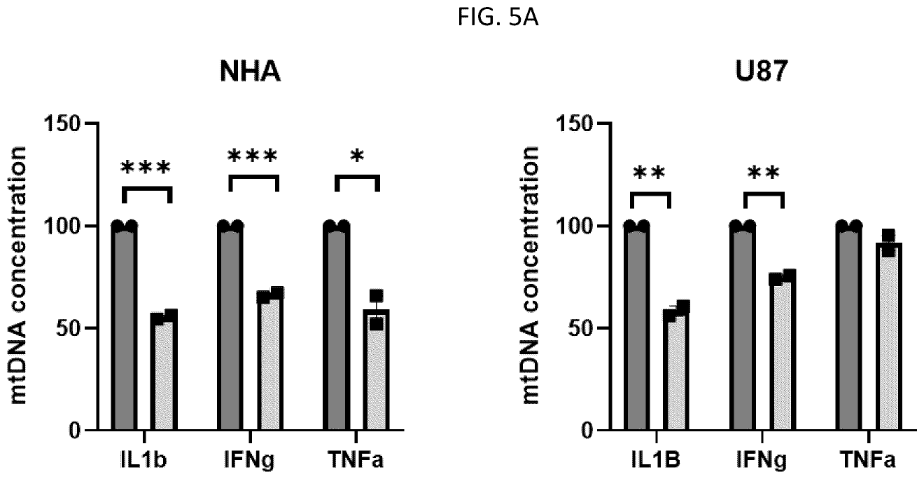

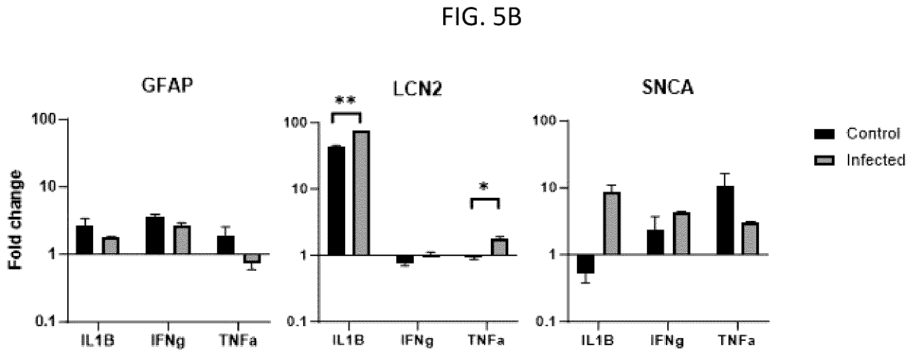

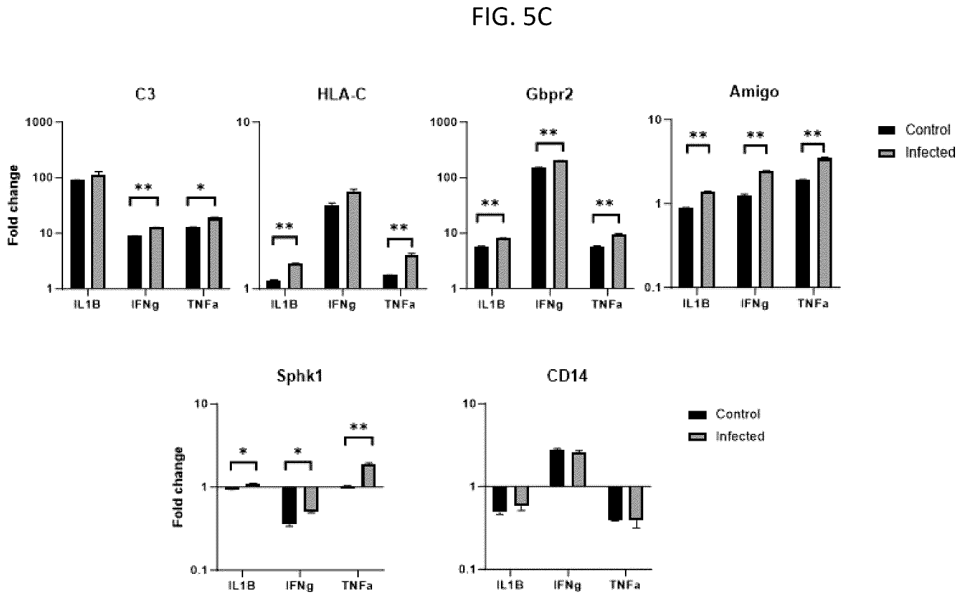

As previously stated, iPSC-derived PD astrocytes show increased expression of LCN2 and GFAP following exposure to inflammatory conditions. Inflammation can additionally induce A1 phenotypic skewing in astrocytes.⁴ Therefore, the analysis of mtDNA, PD astrocyte-associated genes, and A1/A2 skewing was repeated on NHA that were exposed to the inflammatory cytokine IL1β, IFNγ, or TNFα for 24 hours. mtDNA copy number was reduced in infected cells exposed to cytokines as compared to uninfected cells exposed to cytokines (FIG. 5A; IL1β p=0.0004; IFNγ p=0.001; TNFα p=0.03). Exposure to IL1β induced LCN2 expression in both infected and uninfected cells but the expression was significantly higher in HPgV-infected cells (FIG. 5B; p=0.006). Exposure to TNFα significantly induced expression of LCN2 in infected cells but not uninfected cells (FIG. 5B; p=0.04). GFAP expression was induced by exposure to all cytokines, but similar levels of induction were seen in both infected and uninfected cells (FIG. 5B). SNCA expression was induced 10-fold following exposure to IL1β. SNCA induction was also observed to a lesser extent following exposure to IFNγ and TNFα, with no significant difference between infected and uninfected cells (FIG. 5B). A1 astrocyte genes were all upregulated in HPgV-infected cells as compared to uninfected cells following exposure to TNFα (FIG. 5C). ILIB and IFNγ both significantly increased expression of 3/4 A1 associated genes in infected cells as compared to uninfected cells (FIG. 5C).

### REFERENCES

1. Sonninen T M, Hämäläinen RH, Koskuvi M, Oksanen M, Shakirzyanova A, Wojciechowski S, Puttonen K, Naumenko N, Goldsteins G, Laham-Karam N, Lehtonen M, Tavi P, Koistinaho J, Lehtonen S. Metabolic alterations in Parkinson's disease astrocytes. Sci Rep. 2020; 10(1): 14474.
2. Pyle A, Anugrha H, Kurzawa-Akanbi M, Yarnall A, Burn D, Hudson G. Reduced mitochondrial DNA copy number is a biomarker of Parkinson's disease. Neurobiol Aging. 2016; 38:216.e7-216.e10.
3. Chen S-H, Kuo C-W, Lin T-K, Tsai M-H, Liou C-W. Dopamine Therapy and the Regulation of Oxidative Stress and Mitochondrial DNA Copy Number in Patients with Parkinson's Disease. Antioxidants. 2020; 9(11):1159.
4. Liddelow S A, Guttenplan K A, Clarke L E, et al. Neurotoxic reactive astrocytes are induced by activated microglia. Nature. 2017; 541(7638):481-487.
5. Yun S P, Kam T-I, Panicker N, et al. Block of A1 astrocyte conversion by microglia is neuroprotective in models of Parkinson's disease. Nature Medicine. 2018; 24(7):931-938.

### Example 3: Human Pegivirus-Infected Glial Cells Affect Neighboring Neurons

PD pathology is characterized by the loss of dopaminergic (DA) neurons and the accumulation of insoluble α-synuclein (SNCA) deposits. The inventors have determined that PD brains have a high prevalence of HPgV, that astrocytes are the primary infected cell type, and that HPgV does not infect neurons. However, it is well-established that neurons require trophic support from astrocytes to maintain viability, proper synaptic function, and homeostasis.

Thus, in the following example, the inventors demonstrate that SHSY5Y neuroblastoma cells exhibit decreased viability when exposed to HPgV-infected normal human astrocytes (NHA) and increased SNC deposition when exposed to HPgV-infected U87 mg glioblastoma cells. These findings suggest that HPgV-infected glial cells affect nearby neurons.

### Results:

### SHSY5Y Cell Viability Following Exposure to HPgV-Infected NHA

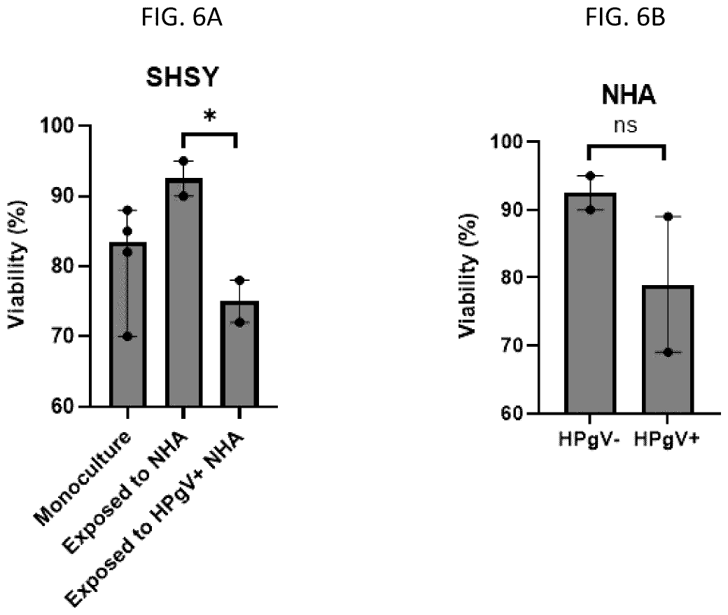

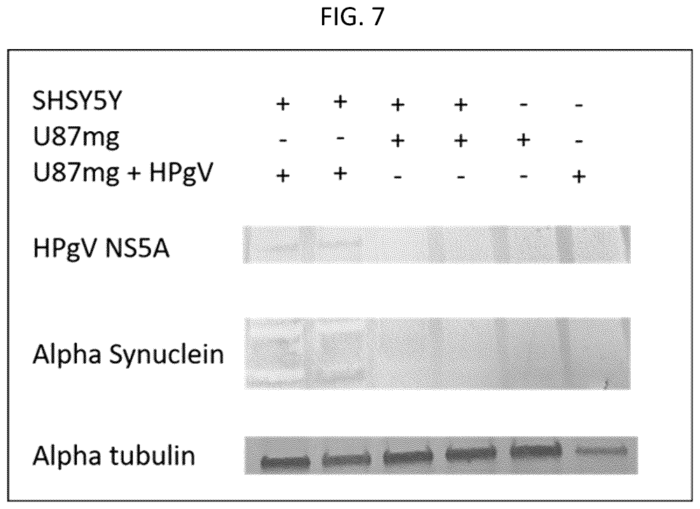

A key component in assessing the role of HPgV-infected glia in PD etiology is to show that HPgV-infected glia can provoke the loss of DA neurons. SHSY5Y cell lines are used to model DA neurons in PD models. Differentiated SHSY5Y cells were exposed to HPgV-infected and uninfected NHA in a co-culture model. The SHSY5Y cells showed a significant reduction in viability after 7 days of exposure to HPgV-infected NHA as compared to uninfected NHA (FIG. 6A; p=0.046; FIG. 7.1). No statistical change in viability was observed in the infected NHA as compared to the uninfected NHA (FIG. 6B).

### Expression of SNCA Protein

Immunoblots were performed to determine whether exposure to HPgV-infected glial cells increases the expression and deposition of SNCA in SHSY5Y cells. SHSY5Y cells that were co-cultured with infected NHA and uninfected NHA both showed similar amounts of SNCA as SHSY5Y cultured alone (data not shown). However, in co-culture with HPgV-infected U87 mg cells, differentiated SHSY5Y showed increased levels of SNCA protein as compared to SHSY5Y cells co-cultured with uninfected U87 mg cells (FIG. 7).

### Example 4: Materials and Methods

In the following example, the inventors describe the materials and methods utilized in Examples 1-3.

### Study Cohort

Parkinson's disease patient and control samples were obtained from the Rush Alzheimer's Disease Center (RADC). Participants were enrolled in the Religious Orders Study/Memory and Aging Project (ROSMAP) study hosted by RADC. PD patients were matched to non-PD control patients based on age, sex, and level of education. PD and non-PD subjects additionally showed similar low levels of Alzheimer's disease pathology, as determined by Braak and CERAD scores which measure the severity of neurofibrillary tangles and neuritic plaque density, respectively.¹ All 10 of the PD cases, but none of the non-PD controls, were considered qualitatively positive for PD pathology according to RADC criteria, which was defined as nigral loss of neurons in the presence of Lewy bodies (i.e., insoluble deposits of SNCA).² Findings of Lewy body dementia were more frequent in the PD cases than in non-PD controls. All subjects had mild (Cognitive DX=2-3) or no (Cognitive DX=1) cognitive impairment, with more cognitive impairment in the PD cases than in the non-PD controls. RADC patient information summarized in Table 3.

Brain tissue samples were obtained from the amygdala (AMG), posterior putamen (PPUT), and superior frontal cortex (SFC) of each study subject, along with formalin fixed paraffin embedded (FFPE) contralateral tissue from the same sample areas, which was sliced to 20 μm thickness. Additionally, plasma and CSF samples were obtained from all study subjects. Brain tissue samples, plasma, and CSF were stored at −80° C. prior to nucleic acid extraction, and DNA and RNA were stored at −80° C. following extraction.

TABLE 3
Demographics of RADC cohort

| | | | | | P- |
|---|---|---|---|---|---|
| | PD | | Control | | value |
| Age (Mean [1 SD]) | 87.2 | (5.1) | 89.9 | (3.3) | 0.16 |
| Female n (%) | 4 | (40%) | 9 | (64%) | 0.48 |
| Years Education (Mean | 16 | (3.3) | 18 | (3.1) | 0.685 |
| [1 SD] | | | | | |
| Post-Mortem | 7.6 | (9.2) | 6.4 | (3.9) | NS |
| Interval (Median [IQR]) | | | | | |
| Pathology | | | | | |
| Braak (Mean [1 SD]) | 2.9 | (1.0) | 3.2 | (0.6) | 0.40 |
| CERAD (Median [IQR]) | 4 | (0) | 4 | (0) | NS |
| PD Pathology n (%) | 10 | (100%) | 0 | (0%) | <0.001 |
| Lewy Body Disease n (%) | | | | | |
| None | 0 | (0%) | 12 | (85.7%) | <0.001 |
| Limbic Type | 4 | (40%) | 0 | (0%) | <0.001 |
| Neocortical Type | 6 | (60%) | 2 | (14.3%) | <0.001 |
| Cognitive DX (Mean | 1.6 | (0.9) | 1.0 | (0) | 0.02 |
| [1 SD]) | | | | | |

### ViroFind

One major limitation to determining viral involvement in neurodegenerative disorders is that screening for viral infections historically required a priori knowledge of candidate viruses. While next generation sequencing (NGS) can be used to identify fulminant infections in which viral genomes far out-number host genomes, this kind of analysis misses most viral species in solid organ samples, in which host nucleic acids far outnumber viral nucleic acids. ViroFind was developed to address these shortcomings.

ViroFind is an in-house, unbiased, targeted enrichment, next-generation sequencing assay that provides improved viral detection via hybridization to biotinylated RNA probes designed to provide nearly full-length genome coverage for all viral species known to infect humans. See Chalkias et al.³, which is hereby incorporated by reference in its entirety.

DNA and RNA were extracted from solid tissue samples with the QIAGEN DNeasy and RNeasy extraction kits, respectively. RNA was processed through a QIAshredder for homogenization. Total nucleic acid was extracted from fluid samples with the MinElute Virus spin kit (QIAGEN, Inc.). Extracted RNA was reverse transcribed with NEBNext first strand synthesis module followed by NEBNext second strand synthesis module. 500 ng each of DNA and cDNA were pooled and sonicated to 150 bp length fragments with the Covaris M220 focused ultrasonicator. Samples were then prepared for sequencing with the Agilent SureSelect XT2 Target Enrichment System for Illumina sequencing as follows: sheared DNA and cDNA were enzymatically end repaired, A-tailed, and ligated to barcoded indexing adaptors. Indexed fragments were then amplified via five rounds of PCR. Samples were purified with AMPure XP beads following end repair, ligation, and amplification. Indexed amplicons were analyzed on the Agilent TapeStation 4200 to obtain the concentration and ensure proper sizing before 187.5 ng of each sample was pooled and concentrated to 3.5 μL using the Savant SPD1010 SpeedVac. The pooled library was then hybridized with ViroFind probes for 24 hours at 65° C. ViroFind probes are biotinylated 125 nucleotide long RNA probes designed against the full-length genome sequences of all viruses known to infect humans as well as viruses considered to have zoonotic potential, with regions of high homology to the human genome removed to yield a median of 89% genome length coverage of all viral species. RNA:DNA hybrids were then bound to magnetic streptavidin beads and unhybridized host DNA and cDNA were washed away. Pooled hybrid DNA was then amplified for an additional 12 rounds by PCR. Amplicons were again cleaned by AMPure bead purification, quantified, and sequenced using Illumina NextSeq.

Each sample produced about 20 million paired-end (PE) 150 bp reads. Raw demultiplexed reads were processed through the ViroFind analysis pipeline v2.0. First, reads with overall quality <20 and length <50 bp were discarded using Skewer (‘-q 20-1 50’).⁴ Reads were further processed to remove low complexity reads and duplicate reads using PRINSEQ++(‘-lc_entropy 90 -derep’).⁵ Reads passing STAR allowing a maximum of 1000 alignments per read (--outFilterMultimapMax 1000).⁶

PE reads that did not map to the Hg38 version of the human genome were mapped against the set of viral reference genomes downloaded from NCBI using STAR using the filter specified above. Reads mapping to multiple viruses were not used for viral identification. For all identified viruses, breadth and depth of coverage were evaluated using BEDTOOLS genomecov.⁷ Sequence Alignment Map (SAM) and its binary version (BAM) files were generated for visualization of the virus aligned regions.⁸ Identified viral regions were matched with gene descriptions from General Feature Format (GFF) files corresponding to the viral references downloaded from NCBI using BEDOPS suite and in-house scripts.⁹ Tab-delimited summary files were generated on a per-sample basis to summarize the identified viruses along with their breadth and depth of coverage, coverage normalized for 1M reads and 1000 bp of genome, viral regions and corresponding genes.

PICARD tools was used to mark and remove PCR-derived duplicate reads to generate a set of unique viral reads for variant calling. V-phaser 2 was used to identify viral variants from the virus aligned reads.¹⁰ Whole genome coverage plots showing per-base coverage across the whole genome for each identified virus were generated using in-house R scripts.

Finally, the reads from identified viruses were assembled into larger contiguous sequences (contigs) using SPAdes de novo assembler.¹¹ Consensus sequence, FASTA and FASTQ files were generated for reads mapping to the viruses using SAMTOOLS.⁸ Results for multiple samples were visualized using complex heatmaps generated with in-house R scripts.¹²

qPCR Assay

An in-house quantitative PCR (qPCR) assay was developed to detect HPgV nucleic acid since no commercial assays are available. The assay was designed to target two genomic regions with degenerate primers and triplicate degenerate TaqMan probes so that diverse viral sequences can be detected. Primer and probe sequences are listed in Table 4. Primer pairs were validated independently using a full genome-length molecular clone obtained from the NIH HIV Reagent Program (Catalog #ARP-9450).¹³ The molecular sequence clusters with genotype 2 HPgV sequences and is contained within cloning vector pCR2.1. The plasmid was spiked into human DNA and each primer pair was tested independently in a standard PCR reaction to ensure that no non-specific amplification occurred. This was then repeated with the addition of degenerate primers. All amplified products were analyzed using the Agilent TapeStation 4200. The primer pairs were then similarly validated using an RNA template obtained through transcription of the full-length HPgV genome by linearization with restriction enzyme SpeI (New England Bio Labs), followed by transcription using the T7 RiboMAX expression system (Promega, Inc.). Resulting genomic RNA (gRNA) was spiked into extracted human DNA and the amplifications were repeated using TaqMan Fast Virus 1-step master mix, which includes a reverse transcriptase step and confirmed that amplification from an RNA template was successful and that no off-target bands were present. The assay was then validated for qPCR on the QuantStudio 5 (ThermoFisher) with the addition of TaqMan probes. HPgV RNA was diluted to 5 ng/μL, which is the equivalent of 1×10⁹ cp/μL. Serial 10-fold dilutions were then used to obtain a linear range between 1×10⁵ and 100 copies per reaction.

TABLE 4
qPCR primers and probes

| Primer | Sequence |
|---|---|
| NS3-F1 | GTGACCTGGTATGGAAT |
| | GGAACCT |
| | (SEQ ID NO: 5) |
| NS3-F2 | GTAACCTGGTATGGAA |
| | TGGAACCT |
| | (SEQ ID NO: 6) |
| NS3-R | CGACGGCTGCGGTGT |
| | AAG |
| | (SEQ ID NO: 7) |
| 5′ UTR-F | TGTTGGCCCTACCG |
| | GTGTTA |
| | (SEQ ID NO: 8) |
| 5′ UTR-R | CCGTACGTGGGCGT |
| | CGTT |
| | (SEQ ID NO: 9) |
| Probe | Detector and Sequence |
| NS3 FAM | 6FAM-AGCTAACCTA |
| | TTGAGACTTTACGAC |
| | GACT-QSY |
| | (SEQ ID NO: 10) |
| NS3 VIC | VIC-AGCTAACCTAC |
| | TGAGACTTTACGACG |
| | ACT-QSY |
| | (SEQ ID NO: 11) |
| NS3 ABY | ABY-AGCTAACCTTC |
| | TGAGACTTTACGACG |
| | ACT-QSY |
| | (SEQ ID NO: 12) |
| 5′ UTR | FAM6FAM-CTCGTCG |
| | TTAAACCGAGCCCGT |
| | CA-QSY |
| | (SEQ ID NO: 13) |
| 5′ UTR VIC | VIC-CTCGTCGTTAA |
| | ACCGAGACCGACA-Q |
| | SY |
| | (SEQ ID NO: 14) |
| 5′ UTR ABY | ABY-CTCGTCGTTAA |
| | ACCGAGACCGTCA-Q |
| | SY(SEQ ID NO: 15) |

Following validation of the assay conditions, qPCR screening was performed for three replicates of each primer and probe set using 10 μL/reaction of extracted RNA (RNeasy, QIAGEN) from tissue and cell samples or 10 μL/reaction of extracted nucleic acid (MinElute, QIAGEN) from cell-free fluid samples in TaqMan Fast Virus 1-step master mix. Cycling conditions were 50° C. for 10 minutes for reverse transcription, 95° C. for 20 seconds to inactivate RT enzyme and denature template, followed by 40 cycles of amplification at 95° C. for 15 seconds and 60° C. for 1 minute. Samples were considered positive if both NS3 and UTR had an average cycle threshold of 38 or less for at least one detector.

### Immunofluorescence Staining

FFPE sections were deparaffinized with xylene for 3 minutes, then washed twice with 100% ethanol (EtOH), followed by washes with 95%, 70%, and 50% EtOH for 3 minutes per wash, followed by rinsing with H₂O. Epitope retrieval was performed for 15 minutes at 125° C. and then 5 minutes at 60° C. in sodium citrate buffer at pH 6 in a BioCare Medical decloaking chamber. Slides were blocked for 1 hour in 5% donkey serum, followed by three 1-minute washes in Dulbecco's PBS (DPBS) with tween 20. Primary antibodies were diluted in DPBS with 2% horse serum and incubated overnight at 4° C. Slides were again washed three times for 1 minute each in PBS with Tris before the addition of secondary antibodies diluted in DPBS with 2% horse serum. Secondary antibody was washed off following a 1-hour incubation at room temperature in the dark. Mounting medium including DAPI was used to counterstain nuclei. Slides were imaged using a Leica DM6000 fluorescence microscope.

### ELISA

HPgV IgG seropositivity was qualitatively determined using the Human Anti-Hepatitis G virus antibody (IgG) ELISA made by LSBio (Catalog #LS-F10271; LifeSpan BioScience, Inc.), which targets HPgV E2 IgG. Samples were run in duplicate according to the manufacturer's protocol, using a Capp Wash 8 hand-held plate washer, and the Synergy HTX plate reader. Samples were determined to be seropositive if the average of the two obtained optical density (OD) values were >0.1 more than the average OD of the negative control.

### Cell Culture

Peripheral blood mononuclear cells (PBMCs): Fresh PBMC were obtained in sodium heparin tubes and isolated using Ficoll density separation. Cells were plated at a density of 4×10⁶ cells/well in a 6-well plate in RPMI supplemented with 10% FBS, 1% penicillin/streptomycin, and 0.01% gentamicin (RPMI-complete). PBMCs were stimulated with 20 IU/mL IL-2, 4 ug/mL LPS, and 5 ug/mL PHA for the first 48 hours of culture and were subsequently maintained with 20 IU/mL IL-2 and 5 ug/mL PHA with medium changes. Each well was fed with 1×10⁶ freshly isolated and stimulated PBMCs every 7 days along with a medium change.

Normal human astrocytes (NHA): Cryopreserved NHA were obtained from Lonza. NHA were maintained in Astrocyte Basal Medium with Astrocyte Growth Medium SingleQuot supplements (Lonza). NHA were seeded at 5×10³ cells/cm and allowed to reach confluency before splitting and seeding into new cell culture vessels, which took approximately 10 days. Medium was changed 24 hours after seeding and every subsequent 48 hours. NHA were discarded at passage 10.

U87 mg: Cryopreserved U87 mg glioblastoma cells were maintained in Eagle's Minimum essential medium (EMEM; ATCC), supplemented with 10% FBS, 1% penicillin/streptomycin, and 0.01% gentamicin. U87 mg were subcultured every 3-4 days at a 1:5 ratio based on confluency, and cultures were terminated after 28 days when cell clumping and detachment were apparent.

iPSC GBA PD neurons: Induced pluripotent stem cells (iPSCs) were differentiated into floorplate precursor cells and subsequently to midbrain dopaminergic neurons through sequential addition and withdrawal of morphogens as previously described.¹⁴ To minimize variability in neuralization cells were scraped into 1-2 mm chunks and passaged onto poly-D-lysine (PDL) and laminin coated plates on day 13. Cells were passaged again via Accutase® (cell detachment reagent) dissociation at day 25 and were replated as single cells on PDL/laminin coated plates. On day 40, neuralization growth factors were removed and the media was replaced with Neurobasal-A (GIBCO) supplemented with Neurocult SM1 (StemCell Technologies) (Neurobasal-Complete). Subsequent medium changes were 50% to maintain trophic factors produced by iPSC DA neurons. Adsorption of HPgV occurred at day 45, and culture was terminated 10 days later.

SHSY5Y: Cryopreserved SHSY5Y neuroblastoma cells were obtained from ATCC. SHSY5Y cells were maintained in RPMI 1640 (Sigma Aldrich) supplemented with 10% FBS, 1% penicillin/streptomycin, and 0.01% gentamicin. Cells were subcultured at a 1:5 ratio when confluent. Cells were differentiated with the addition of 10 μM all trans retinoic acid (RA; Sigma Aldrich) every 48 hours for 5 days before use, as previously described.¹⁵

### Viral Stock Preparations, Transfection, and Infection

Infectious HPgV RNA was prepared by transcription from a full-length molecular clone obtained from the NIH HIV Reagent Program (Catalog #ARP-9450) as previously described.^{13,16} Briefly, the cloning plasmid was amplified by transduction into competent DH5a E. coli, isolated with QIAGEN Miniprep spin column, linearized with SpeI restriction enzyme digestion (New England Biolabs), purified by EtOH precipitation, transcribed using the T7 RiboMax large scale transcription kit (Promega, Inc.), and cleaned up with Phenol:chloroform.

Transfection into PBMC was performed using the JetMessenger transfection kit (PolyPlus) according to manufacturer's protocol using 1 μg of RNA per well. Cells were washed with DPBS after 24 hours and RPMI-Complete was replaced.

Infection was performed using supernatant from infected cells. PBMC inoculum was used to infect the initial NHA cultures and U87 mg culture, while subsequent infectious inoculum was produced in U87 mg cells. Infection was performed for NHA, U87 mg, and SHSY5Y by adsorption with fresh inoculum for 5 hours before washing with DPBS and replacing medium. Infection in iPSC-derived DA neurons was performed by diluting inoculum to 50% with pre-conditioned medium, which was then adsorbed for 5 hours before washing with DPBS and replacing 50% pre-condition medium with 50% Neurobasal-complete medium.

HCMV-GFP viral inoculum¹⁷ was adsorbed for 5 hours, washed with DPBS, and medium was replaced. HCMV infected cells were monitored daily for expression of GFP and terminated when 50% of cells expressed GFP at day 7 post infection.

qPCR Analysis of Host Genes

Quantitation of mitochondrial DNA (mtDNA) copy number was performed on DNA extracted from cell pellets using the DNeasy spin column kit (QIAGEN) according to manufacturer's protocol. DNA was quantitated using the Qubit BR dsDNA kit and diluted to a 5 ng/μL working concentration. qPCR was then performed in triplicate for both CYTB and APP using 25 ng of template DNA in 2×SYBR green PCR master mix (Applied Biosystems, Inc) in a 25 μL reaction. qPCR was performed on the QuantStudio 5 using a 10-minute incubation at 95° C. followed by 40 cycles of a 15-second denaturation at 95° C. and a 1-minute extension at 60° C., after which melt curve analysis was performed. mtDNA concentrations were compared by ΔΔCT, which was calculated as the average of the cycle threshold for CYTB (mitochondrial gene) amplification minus the average of the cycle threshold for APP (nuclear gene) amplification for the infected cells relative to the uninfected cells.

Quantification of A1/A2 genes was performed on RNA from pelleted cells using the RNeasy spin column kit (QIAGEN) with β-mercaptoethanol added to lysis buffer and using a QIAshredder for sample homogenization, according to manufacturer's protocol. Extracted RNA was eluted using 90 μL of nuclease-free H₂O. Extracted RNA was then treated using recombinant DNAse I (NEB) for 1 hour at 37° C. DNAse was inactivated at 65° C. for 10 minutes, and then isopropanol precipitation was performed overnight at −80° C. Precipitated RNA was pelleted for 1 hour at 4° C. at 15,000 RPM, supernatant was removed, and the pellet was washed with freshly made, cold, 70% ethanol. Pellets were re-spun for 15 minutes at 4° C. and supernatant was removed, and then the pellets were allowed to air-dry in a biosafety cabinet in the dark for 30 minutes at room temperature. RNA pellets were then resuspended in 50 μL of nuclease-free H₂O and quantitated with the Qubit BR ssRNA kit. 500 ng of total RNA was reverse transcribed with SuperScript III using random hexamers for 45 minutes at 50° C., followed by heat inactivation and RNAse H treatment. Samples were then ethanol precipitated as previously described and resuspended in 70 μL of nuclease-free H₂O. SYBR green qPCR was performed and analyzed for duplicate reactions for each primer set using 5 μL of cDNA template in a 25 μL volume reaction, as previously described.

Quantification of PD astrocyte associated gene expression was performed on similarly extracted RNA, following DNAse digestion and isopropanol precipitation. DNAse-treated RNA was resuspended in 50 μL of nuclease-free H₂O, quantitated using the Qubit BR ssRNA kit, and diluted to 40 ng/μL. Commercially available TaqMan gene expression assays against GFAP (#Hs00909233; Fisher Scientific), LCN2 (#Hs01008571), SNCA (#Hs00240906), and ITPR2 (#Hs00181916), using FAM as a detector, were each multiplexed with GAPDH (housekeeping gene; #Hs02786624) using VIC as a detector. Each assay was run in duplicate 20 μL reactions, with 5 μL of diluted template RNA, 1 μL of each appropriate 20× assay, and 5 μL of TaqMan Fast Virus 1-step master mix. ΔΔCT was calculated as previously described. All primer sequences are listed in Table 5.

TABLE 5
mtDNA and A1/A2 Primers

| Primer | Sequence |
|---|---|
| APP F | GCCTGCCTGATCCTCCAAAT (SEQ ID NO: 16) |
| APP R | AGGGTAGCGGATGATTCAGCC (SEQ ID NO: 17) |
| CYTB F | TGTGTGCTCTCCCAGGTCTA (SEQ ID NO: 18) |
| CYTB R | CAGTTCTGGATGGTAACTGG (SEQ ID NO: 19) |
| C3 F | GACATTCCGGAACTCGTCAA (SEQ ID NO: 20) |
| C3 R | CGTACTCCTTCACCTCAAACTC (SEQ ID NO: 21) |
| HLA-CF | CTGGACAAGAGCAGAGATACAC (SEQ ID NO: 22) |
| HLA-CR | AGCAACGATGCCCATGAT (SEQ ID NO: 23) |
| Gbp2 F | GTTGTGCATCCGAAAGTTCTTC (SEQ ID NO: 24) |
| Gbp2 R | GGTTCAGCTCTTCCTCCTTTAG (SEQ ID NO: 25) |
| Amigo F | AGGTACTTCTGCTCCAGGATAG (SEQ ID NO: 26) |
| Amigo R | CGACCTGAGCCTCATGAATAAA (SEQ ID NO: 27) |
| Sphk1 F | GGCGTCATGCATCTGTTCTA (SEQ ID NO: 28) |
| Sphk1 R | CACGGGCACATATACCAAGTAG (SEQ ID NO: 29) |
| CD14 F | GCTGGAACAGGTGCCTAAA (SEQ ID NO: 30) |
| CD14 R | CCGTCCAGTGTCAGGTTATC (SEQ ID NO: 31) |
| GAPDH F | TGACTTCAACAGCGACACCCACT (SEQ ID NO: 32) |
| GAPDH R | ACCACCCTGTTGCTGTAGCCAAAT (SEQ ID NO: 33) |

### Immunoblots

Cells were washed 1× with sterile DPBS, then gently lifted from cell culture plates using a cell scraper in 1 mL of DPBS. Cells in DPBS were then transferred into a 1.5 mL microcentrifuge tube and pelleted for 5 minutes at 300×g at room temperature, after which the supernatant was removed. Cell pellets were then resuspended in 200 μL RIPA buffer supplemented with complete Protease Inhibitor Cocktail (1 tablet into 10 mL RIPA; Roche Diagnostics). Each resuspended pellet was then sonicated for 15 seconds at a steady amplitude of 30 using a Q700 ultra sonicator (QSonica) and was then incubated on ice for 30 minutes to allow for lysis. Lysed samples were then pelleted at 13,000 RPM in a microcentrifuge for 10 minutes at 4° C. Pelleted samples were then either processed via western blot or stored at −20° C. Protein concentration was determined using the Qubit Protein Assay following the manufacturer's directions. 14 μg of extracted protein was then diluted into 12.5 μL of 2× Laemmli buffer and brought to 25 μL total volume with RIPA buffer. Diluted samples were boiled for 5 minutes and were then loaded into a 4-20% Mini-PROTEAN TGX Gel (Bio-Rad, Inc.) and run in Tris/Glycine/SDS buffer (Bio-Rad, Inc.) for 1 hour at 100 cv against the iBright prestained protein ladder (Invitrogen, Inc.). Separated proteins were then transferred to PVDF membranes using the iBlot gel transfer device (Invitrogen, Inc.). Transferred blots were blocked in 5% bovine serum albumin (BSA) for 30-60 minutes and were then incubated with diluted primary antibody overnight at 4° C. NHA blot was then detected using the Fast Western Blot ECL Substrate kit (Pierce Biotechnology), according to the manufacturer's protocol. U87 mg blot was detected using the Vectastain Elite ABC Universal Peroxidase Kit (Vector Laboratories), and DAB substrate Kit (Vector Laboratories), according to the manufacturer's protocol. Western blots were imaged using the iBright imaging system (Invitrogen, Inc).

### Statistical Analysis

Comparisons between two groups were performed by unpaired Student's T test using GraphPad Prism 9 (GraphPad Software). Comparisons between proportions of two variables were performed by Fisher's Exact test through GraphPad Prism 9. Comparisons between nonparametric data between two groups was performed by Mann-Whitney using GraphPad Prism 9. Significance was determined for two-tailed p-values of <0.05. False Discovery Rate q-values, Normalized Enrichment Scores, and p-values associated with RNAseq data are intrinsic functions of the analysis pipelines stated.

### REFERENCES

1. Bennett D A, Schneider J A, Arvanitakis Z, et al. Neuropathology of older persons without cognitive impairment from two community-based studies. Neurology. 2006; 66(12):1837-1844.
2. Buchman A S, Yu L, Wilson R S, et al. Progressive parkinsonism in older adults is related to the burden of mixed brain pathologies. Neurology. 2019; 92(16):e1821-e1830.
3. Chalkias S, Gorham J M, Mazaika E, et al. ViroFind: A novel target-enrichment deep-sequencing platform reveals a complex JC virus population in the brain of PML patients. Plos one. 2018; 13(1):e0186945.
4. Jiang H, Lei R, Ding S W, Zhu S. Skewer: a fast and accurate adapter trimmer for next-generation sequencing paired-end reads. BMC Bioinformatics. 2014; 12; 15:182.
5. Cantu V A, Sadural J, Edwards R. PRINSEQ++, a multi-threaded tool for fast and efficient quality control and preprocessing of sequencing datasets. PeerJ; 2019.
6. Dobin A, Davis C A, Schlesinger F, Drenkow J, Zaleski C, Jha S, Batut P, Chaisson M, Gingeras T R. STAR: ultrafast universal RNA-seq aligner. Bioinformatics. 2013; 29(1): 15-21.
7. Quinlan A R, Hall I M. BEDTools: a flexible suite of utilities for comparing genomic features. Bioinformatics. 2010; 26(6):841-2.
8. Li H, Handsaker B, Wysoker A, Fennell T, Ruan J, Homer N, Marth G, Abecasis G, Durbin R; 1000 Genome Project Data Processing Subgroup. The Sequence Alignment/Map format and SAMtools. Bioinformatics. 2009; 25(16):2078-9.
9. Neph S, Kuehn M S, Reynolds A P, Haugen E, Thurman R E, Johnson A K, Rynes E, Maurano M T, Vierstra J, Thomas S, Sandstrom R, Humbert R, Stamatoyannopoulos J A. BEDOPS: high-performance genomic feature operations. Bioinformatics. 2012; 28(14):1919-20.
10. Yang X, Charlebois P, Macalalad A, Henn M R, Zody M C. V-Phaser 2: variant inference for viral populations. BMC Genomics. 2013; 14:674.
11. Bankevich A, Nurk S, Antipov D, Gurevich A A, Dvorkin M, Kulikov A S, Lesin V M, Nikolenko S I, Pham S, Prjibelski A D, Pyshkin A V, Sirotkin A V, Vyahhi N, Tesler G, Alekseyev M A, Pevzner P A. SPAdes: a new genome assembly algorithm and its applications to single-cell sequencing. J Comput Biol. 2012; 19(5):455-77.
12. Gu Z, Eils R, Schlesner M. Complex heatmaps reveal patterns and correlations in multidimensional genomic data. Bioinformatics. 2016; 32(18):2847-9.
13. Xiang J, Wünschmann S, Schmidt W, Shao J, Stapleton J T. Full-length GB virus C (Hepatitis G virus) RNA transcripts are infectious in primary CD4-positive T cells. Journal of virology. 2000; 74(19):9125-9133.
14. Kriks S, Shim J-W, Piao J, et al. Dopamine neurons derived from human ES cells efficiently engraft in animal models of Parkinson's disease. Nature. 2011; 480(7378):547-551.
15. Pantazopoulou M, Brembati V, Kanellidi A, Bousset L, Melki R, Stefanis L. Distinct alpha-Synuclein species induced by seeding are selectively cleared by the Lysosome or the Proteasome in neuronally differentiated SH-SY5Y cells. Journal of Neurochemistry. 2021; 156(6):880-896.
16. Doan M A L, Roczkowsky A, Smith M, Blevins G, van Landeghem F K H, Gelman B B, Branton W G, Stapleton J T, Hobman T C, Power C. Infection of Glia by Human Pegivirus Suppresses Peroxisomal and Antiviral Signaling Pathways. J Virol. 2021; 95(23):c0107421.
17. Borenstein R, Hanson B A, Markosyan R M, et al. Ginkgolic acid inhibits fusion of enveloped viruses. Scientific reports. 2020; 10(1):4746-020-61700-0.

### Example 5: Repurposing Hepatitis C Virus Drugs to Treat Human Pegivirus Infection

In the following example, the inventors describe their efforts to repurpose hepatitis C virus drugs for the treatment of human pegivirus infections.

### Introduction:

Human pegivirus (HPgV) is a positive, single-stranded RNA virus from the family Flaviviraidae¹. HPgV genome organization is similar to that of hepatitis C virus (HCV), and an estimated 12-37% of the HCV infected population is suspected to also be infected with HPgV-1^{2 3, 4}. Both HCV and HPgV have an open reading frame that is translated into a single pre-polyprotein prior to polypeptide cleavage ^{2, 4}. The pre-polyprotein of HCV is cleaved into two structural proteins (two envelope proteins: E1 and E2) and six non-structural proteins (NS2, NS3, NS4A, NS4B, NS5A, and NS5B) ^{2, 4}. HCV has one extra protein that is not present in HPgV, i.e., the nucleocapsid or core protein ⁴. Cleavage of the non-structural proteins NS2 and NS3 is mediated by NS2, and the cleavage of the other non-structural proteins (i.e., NS4A, NS4B, NS5A, and NS5B) is mediated by NS3 with NS4A as a cofactor⁵⁻⁷. NS4B is a small, hydrophobic protein that possibly assists with membrane-like structure formation ². NS5A is a small, phosphorylated cytosolic protein that is suspected to play a role in RNA replication ². NS5B is an RNA-dependent RNA polymerase that is necessary for viral replication ².

HPgV is typically considered a non-pathogenic, lymphotropic virus, but association with HCV and recent patient cases suggest that HPgV has a more significant clinical role, specifically in immunology and neurology ⁸. HPgV is a blood-borne virus that is commonly transmitted via contaminated blood, intravenous drug use, sexual contact, and mother-to-child transmission ^{3, 9 10 11 12 13, 14 15}. Prevalence of HPgV in North America, Europe, Australia is anywhere between 1.1-6.0% for the healthy blood donor population and 9-48.6% for at-risk donors². In Asia, Africa, and South America, the rate of prevalence is between 0.8-46.6% for healthy donors and 1.8-75.3% for the at-risk population ². Immunocompromised individuals and intravenous drug users are considered the most at risk of becoming infected with HPgV, and some individuals are unable to clear the virus for decades ^{3, 4, 16, 17}.

There is increasing evidence supporting the hypothesis that HPgV infections can, and do, occur in the central nervous system (CNS). We hypothesize that HPgV-1 CNS infections can be treated by repurposing pre-existing HCV antivirals due to the sequence, structure, and viral replication similarities between HPgV and HCV. Seven HPgV infections in the CNS have been reported to date^{18-20 21, 22}. Balcom et al. found two fatal HPgV-1 related encephalitis cases that indicated neurotropism of HPgV-1 ¹⁷. Later, a metagenomic microarray study discovered a novel HPgV infection in brain tissue spinal fluid of a patient with severe encephalitis with no other microbial CNS infections; however, there was uncertainty that the observed clinical symptoms were caused by the HPgV infection ¹⁹. Recently, another four cases of HPgV CSF infection were identified in immunocompromised post-transplant patients²². Next-generation sequencing showed that one of these patients was also HPgV-positive within the brain parenchyma²¹. Patient medical history, clinical symptoms, and radiological similarities between these patients further supports the argument that HPgV has CNS involvement ²².

### Results:

### Consensus Sequence Alignment

The HCV consensus polyprotein and the HPgV consensus polyprotein had 37.74% sequence similarity. The HCV NS3 protein (SEQ ID NO: 1) and the HPgV NS3 protein (SEQ ID NO: 3) had 40.91% sequence similarity. The HCV NS4A protein and the HPgV NS4A protein had 25.53% sequence similarity. The HCV NS4B protein and the HPgV NS4B protein had 29.71% sequence similarity. The HCV NS5A protein and the HPgV NS5A protein had 34.02% sequence similarity. The HCV NS5B protein (SEQ ID NO: 2) and the HPgV NS5B protein (SEQ ID NO: 4) had 36.73% sequence similarity.

### Protein Structure Determination

Ramachandran plots showed low confidence in residue phi and psi angles for NS4A, NS4B, and NS5A in both viruses (data not shown). The MolProbity analysis showed unfavorable protein geometry for these six structures (data not shown). The NS4A, NS4B, and NS5A 3D structures were unstable and were not used for molecular docking studies. The HCV-NS3, HPgV-NS3, and HPgV-NS5B structures did have favorable Ramachandran plots with high confidence in the phi and psi angles of residues and little to no outliers (data not shown). MolProbity predicted favorable protein geometry with little steric clashing among protein residues for HCV-NS3, HPgV-NS3, and HPgV-NS5B (data not shown). The HCV-NS5B SwissModel predicted structure was not stable enough for later docking studies (data not shown). Thus, an x-ray crystallography model (PDB: 3FQK) was used instead ⁵¹. 3FQK has 75.22% sequence identity with our consensus HCV polyprotein sequence²³.

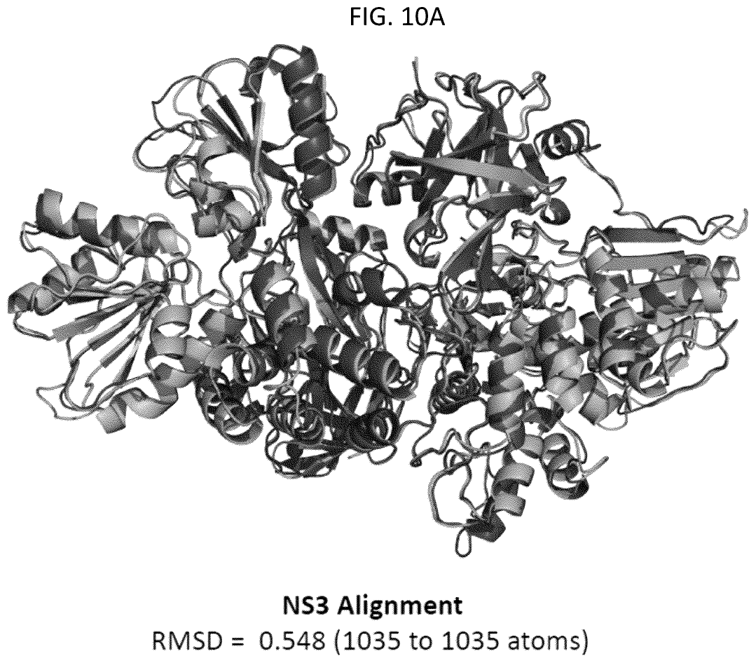

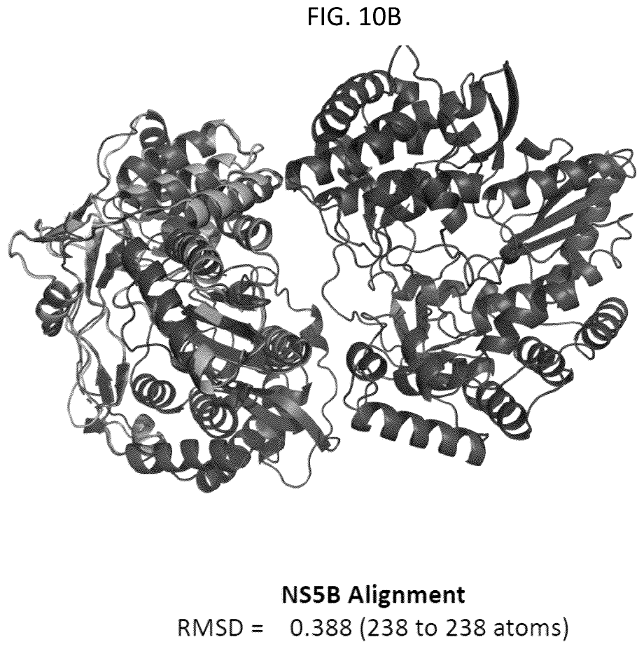

The HCV and HPgV protein structures were aligned in PyMol (FIGS. 10A-10B). The HCV-NS3 and HPgV-NS3 secondary structure locations had a very close alignment with an RMSD of 0.548, where an RMSD<2 is considered favorable (FIG. 10A). HCV-NS5B is about 200 amino acids larger than HPgV-NS5B. Despite the size difference, alignment of these structures showed highly similar locations of secondary structures with an RMSD of 0.388 (FIG. 10B). Molecular docking studies comparing HCV-NS3 to HPgV-NS3 and HCV-NS5B to HPgV-NS5B could proceed based on the sequence and 3D structure similarity of the two viruses.

### Molecular Docking

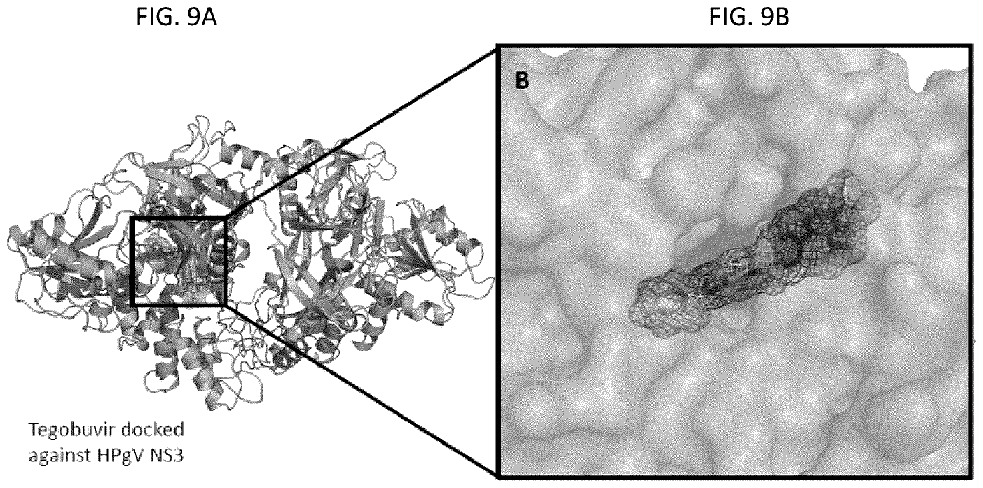

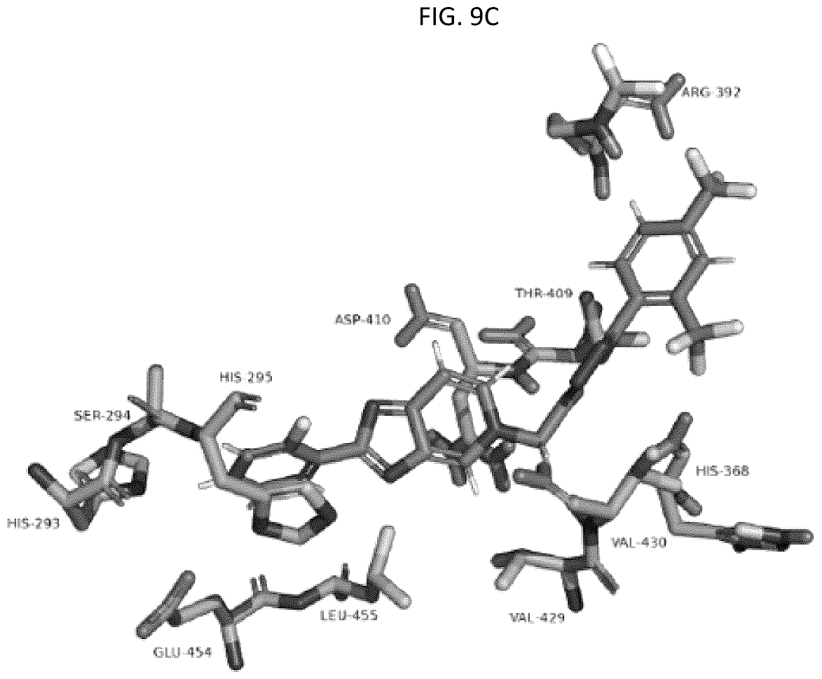

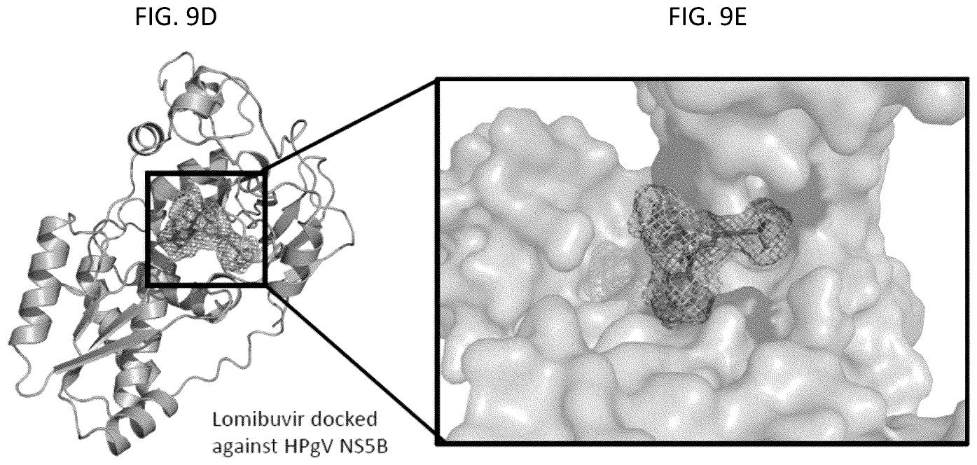

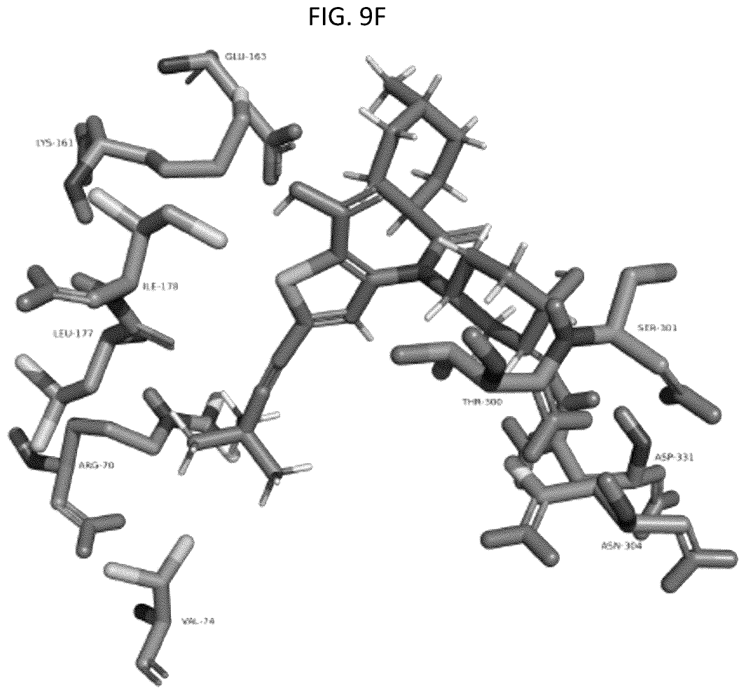

The NS3 protein is responsible for helicase and protease activity in both HCV and HPgV ². Overall, molecular docking resulted in similar GOLD fitness scores (FS) between HCV-NS3 and HPgV-NS3 (Table 6; FIGS. 9A-9F) ³⁶. The FS is a summation of free energy changes of intermolecular forces associated with binding ³⁶. A higher FS indicates stronger potential binding ability and, therefore, greater predicted drug activity. Five compounds (boceprevir, tegobuvir, grazoprevir, danoprevir, and paritaprevir) showed greater predicted binding in HPgV-NS3 than HCV-NS3. The largest FS difference was observed for boceprevir, which favored HPgV by 7.76. Protein-drug interactions also revealed that several HPgV-NS3 amino acids (i.e., HIS293, SER294, HIS295, HIS368, THR409, ASP410, LEU455) were commonly used for drug binding ⁵².

TABLE 6
GOLD molecular docking results

| | | NS | GOLD Fitness Score | | |
|---|---|---|---|---|---|
| | | Protein | | | HCV − |
| Company | Drug Name | Target | HCV | HPgV | HPgV |
| Merck | Boceprevir | NS3 | 54.42 | 62.18 | −7.76 |
| Gilead | Tegobuvir | NS3 | 60.08 | 67.75 | −7.67 |
| Merck | Grazoprevir | NS3 | 63.38 | 69.84 | −6.46 |
| Roche | Danoprevir | NS3 | 67.12 | 72.67 | −5.55 |
| Enata | Paritaprevir | NS3 | 73.31 | 74.20 | −0.89 |
| Pharmaceuticals | | | | | |
| Bristol-Meyer | Asunaprevir | NS3 | 67.37 | 66.02 | 1.35 |
| — | Ro-9187 | NS5B | 35.36 | 34.00 | 1.36 |
| Roche | Mericitabine | NS5B | 54.20 | 51.38 | 2.82 |
| — | 2′-C- | NS5B | 45.54 | 42.60 | 2.94 |
| | methylguanosine | | | | |
| Vertex | Telaprevir | NS3 | 66.19 | 62.66 | 3.53 |
| Pharmaceuticals | | | | | |
| Gilead | Lomibuvir | NS5B | 55.27 | 48.62 | 6.65 |
| | (VX-222) | | | | |
| Janssen | Simeprevir | NS3 | 73.82 | 65.87 | 7.95 |
| Gilead | Psi-7976 | NS5B | 66.11 | 57.32 | 8.79 |
| Gilead | Sofosbuvir | NS5B | 63.67 | 54.79 | 8.88 |
| R-Pharm | Narlaprevir | NS3 | 74.56 | 65.59 | 8.97 |
| AbbVie | Dasabuvir | NS5B | 67.03 | 54.70 | 12.33 |
| Roche | 4′- | NS5B | 48.38 | 34.48 | 13.9 |
| | Azidocvtidine | | | | |
| AbbVie | Glecaprevir | NS3 | 67.74 | 50.68 | 17.06 |
| Vertex | VCH-916 | NS5B | 67.45 | 48.91 | 18.54 |

The NS5B protein is necessary for RNA polymerase activity and is crucial for viral replication ². No HPgV-NS5B docked ligands resulted in a higher FS than the HCV-NS5B docked ligand. Overall, the HCV-NS5B and HPgV-NS5B FS were similar with slightly higher HCV-NS5B scores. Protein-drug interactions of the top 5 compounds revealed that several HPgV NS5B amino acids (i.e., LEU177, SER295, GLY296, THR300, SER301, ASN304, ASP331, VAL297) were repeatedly used for drug binding ⁵².

### Analysis of Top Drugs

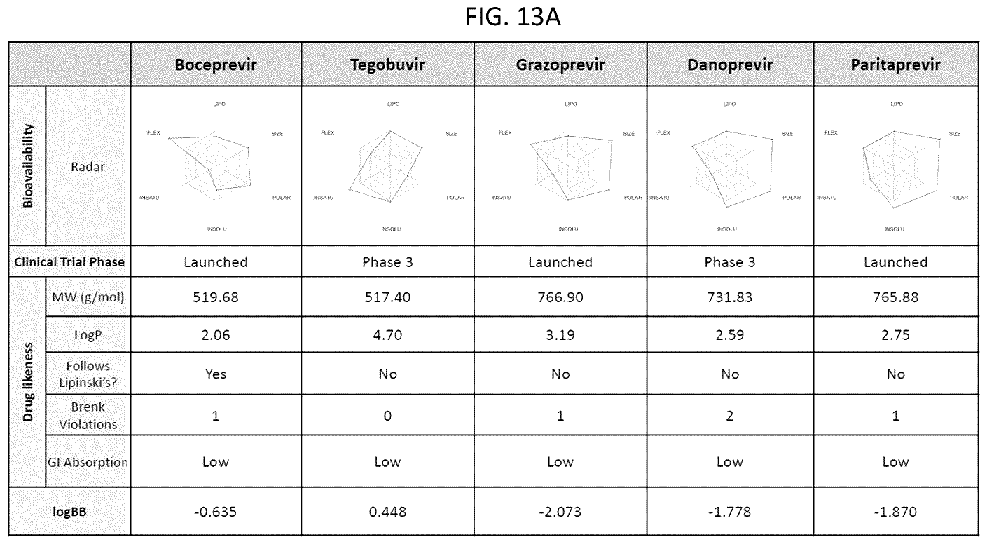

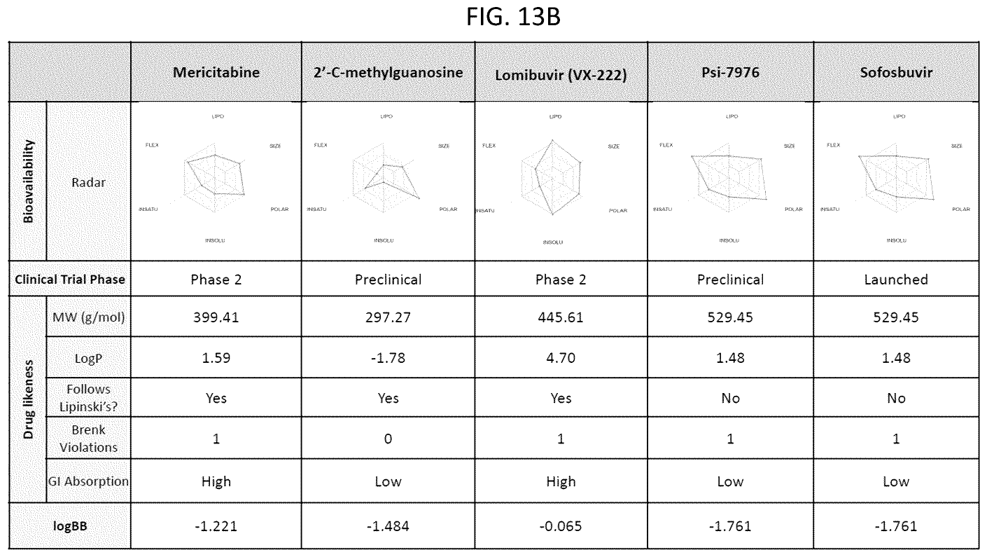

The top five HPgV-NS3 docked compounds were further assessed based on their bioavailability, drug-likeness, and blood-brain barrier (BBB) permeability (FIG. 13A). All 5 compounds had variations from the optimal bioavailability radar, but boceprevir and tegobuvir had the closest preferred physiochemical properties. Additionally, boceprevir is the only NS3 targeting compound that passed Lipinski's Rule of 5^{8, 42}. Tegobuvir has two Lipinski's rule violations, however the violations are close to cut off values for molecular weight and hydrogen bond acceptors, indicating that tegobuvir could still have favorable drug likeness. Tegobuvir is the only NS3 targeting drug to have 0 Brenk Violations and is the only compound in this study with a log BB greater than 0.3 (FIGS. 9A-9C) ^{42, 44}.

The top five HPgV-NS5B docked compounds were mericitabine, 2′-C′-methylguanosine, lomibuvir, PSI-7976, and sofosbuvir (FIG. 13B). Ro-9187 did have the smallest difference in HCV-NS5B and HPgV-NS5B fitness scores; however, this drug was not included for further analysis due to its very low FS in both HCV-NS5B (35.36) and HPgV-NS5B (34.00). Sofosbuvir was the only launched HCV-NS5B drug that was shortlisted for further analysis. PSI-7976 is an isomer of sofosbuvir and, unsurprisingly, the two compounds have similar FSs and physiochemical properties. These two are the only drugs in the top 5 NS5B compounds that do not follow Lipinski's Rule of 5. 2′-C-methylguanosine is the only compound without a Brenk Violation. Lomibuvir was the compound chosen for further molecular dynamics analysis due to its high log BB value in comparison to the other NS5B drugs that are highly unlikely to pass through the BBB (FIGS. 9D-9F).

### Molecular Dynamics

Preliminary data (1 ns simulation time) indicates a stable simulation environment for the HPgV-NS3-tegobuvir complex (data not shown). Radius of gyration (Rg) plots showed protein unfolding as tegobuvir binds (data not shown).

### Discussion:

### Protein Structures

To our knowledge, no NS HPgV crystal structures or 3D homology models have been published. We were able to successfully generate stable HPgV-NS3 and HPgV-NS5B protein models from their amino acid sequences. HPgV-NS3 is nearly identical to HCV-NS3 in size and secondary structure location. HCV-NS5B is roughly 200 amino acids larger than HPgV-NS5B, yet alignment of structures resulted in close secondary structures.

### Molecular Docking

For both NS3 and NS5B, the conformations of docked ligands varied greatly between HCV docked compounds and HPgV docked compounds. Protein-ligand binding interactions showed differences in hydrophobic interactions and hydrogen bonding between HCV and HPgV. The amino acids important for drug binding also varied between HCV and HPgV. The difference in drug-binding interactions is likely explained by the sequence divergence of HCV and HPgV, which also explains some of the variations in FS.

HPgV-NS3 docked ligands had more hydrophobic interactions than HCV-NS3 docked ligands, but HCV-NS3 typically had more hydrogen bonding. The elevated HCV-NS5B FS can be explained by the size difference between NS5B of HCV and HPgV. HCV-NS5B has more residues for drugs to interact with and the ligands docked deeper in the active site groove than in HPgV-NS5B. Despite having lower FSs, HPgV-NS5B docked ligands had more hydrophobic interactions than HCV-NS5B docked ligands.

### Analysis of Top Drugs

The mechanism of action (MOA) for most HCV-NS3 targeting drugs is binding to the NS3 serine protease active site and preventing the cleavage of the HCV polyprotein, which is needed for viral replication 53. Three of the five top NS3 targeting drugs are approved for clinical use to treat HCV infections. Tegobuvir and danoprevir are both in Phase 3 clinical trials. Potential BBB permeability was the most considered factor when choosing a drug for molecular dynamics studies. The size and log BB of grazoprevir, danoprevir, and paritaprevir make them unlikely candidates to cross the BBB. Boceprevir and tegobuvir are the most favorable HPgV-NS3 anti-viral candidates based on their FS, drug likeness, and physiochemical properties. Boceprevir is not predicted to be BBB permeable without modifications to increase lipophilicity. Boceprevir's FS, drug properties, and ready availability make this compound a good potential candidate for inhibiting lymphotropic HPgV infections, but this drug is likely not favorable in a neurological setting and it was not chosen for molecular dynamics simulations. Tegobuvir has slightly less favorable drug-like properties, but it was still chosen for molecular dynamics simulations due to its high log BB score.

The MOA of HCV-NS5B inhibitors varies significantly by drug. Sofosbuvir, for example, is a U nucleotide analog that undergoes several hydrolyzation steps before being incorporated into a primer strand ⁵⁴. This U nucleotide analog results in chain termination, and therefore, inhibits HCV replication ⁵⁴. On the other hand, dasabuvir is a non-nucleoside inhibitor that binds to the allosteric palm domain of NS5B to induce a conformational change that prevents viral elongation ⁵⁵. Sofosbuvir, dasabuvir, mericitabine, and 2′-C′-methylguanosine had low log BB scores and are unlikely to be BBB permeable. Lomibuvir, while not readily able to pass the BBB, has the most potential of NS5B targeting ligands to pass the BBB and was therefore picked for molecular dynamics simulation studies.

### Conclusions:

While HPgV does not currently have well-known clinical symptoms or impacts on health, the association with HCV infection and CNS infections suggests that HPgV likely has more pathogenic effects than previously suspected. This study showed that the HPgV proteins NS3 and NS5B are druggable targets, and that several HCV drugs may be able to inhibit viral replication in vivo. A total of 10 NS3 and 9 NS5B direct acting antivirals (DAAs) were docked against HCV and HPgV proteins. Molecular docking revealed five NS3 targeting drugs with stronger affinity for HPgV than HCV. The remainder of the HPgV-NS3 docked drugs and all the HPgV-NS5B docked drugs had higher HCV affinity to varying degrees. We identified two HCV-NS3 inhibitors, tegobuvir and boceprevir, and one HCV-NS5B inhibitor, lomibuvir, with promising HPgV-NS3 and HPgV-NS5B inhibitory abilities, respectively.

### Materials and Methods:

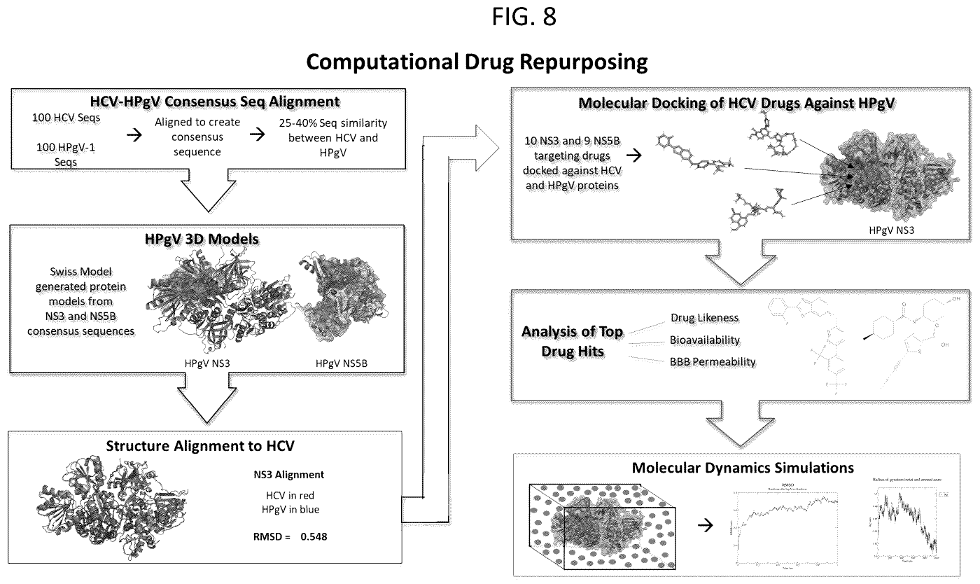

A flowchart representation of the methods is presented in FIG. 8.

### Consensus Sequence Alignment

100 HCV sequences and 100 HPgV-1 sequences were aligned to create the consensus sequences used for the remainder of this study. The sequence similarity between the two viruses was compared using general NCBI protein-protein BLAST parameters ²³.

### Protein Structure Determination

SwissModel, an online software that generates 3D homology models from FASTA sequences, was used to generate protein structures for NS3, NS4A, NS4B, NS5A, and NS5B sequences of both HCV and HPgV (data not shown) ^{24-28 29-32}. The protein template with highest sequence identity, coverage, and QMEANDisCo score for each NS protein was chosen to build the homology model. Protein structural stability was assessed based on Ramachandran plot confidence and MolProbity analysis ³³. Hydrogens were added to the protein file using Molprobity. All suggested asparagine, glutamine, and histidine flips were accepted. The generation of flips allows for more precise estimates of hydrogen bonding and steric interactions to better predict residue clashes ³³. Proteins with Clash scores in the 80th percentile or higher were determined to be suitable for this analysis.

### Drug Selection and Preparation

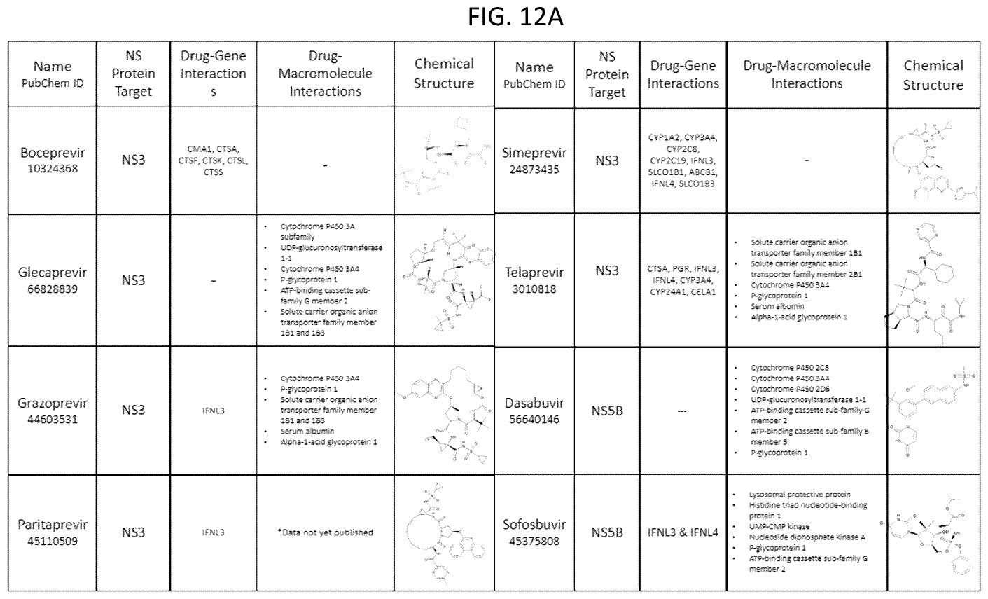

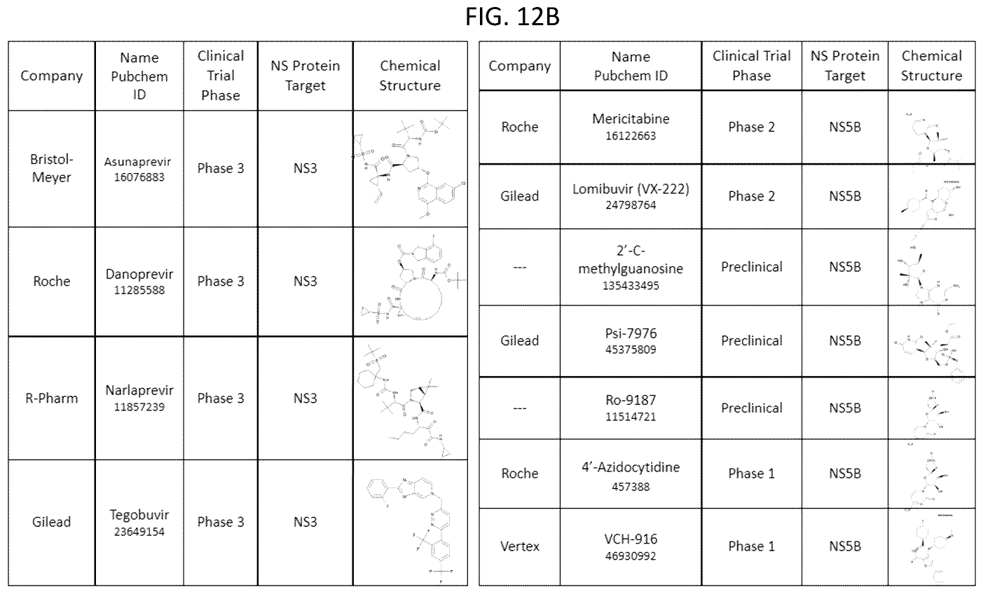

Direct acting antiviral (DAA) drugs against HCV that target the proteins NS3 and NS5B were selected for analysis. The subset of these drugs with FDA approved status is described in FIG. 12A, and the subset that is currently in clinical trials is described in FIG. 12B. 3D drug files were obtained from PubChem, where possible, and were otherwise generated using OpenBabel at physiological pH from Canonical SMILES (2D SMILES file to 3D MDL MOL format, pH 7.4) when not available ^{34,35}

### Molecular Docking

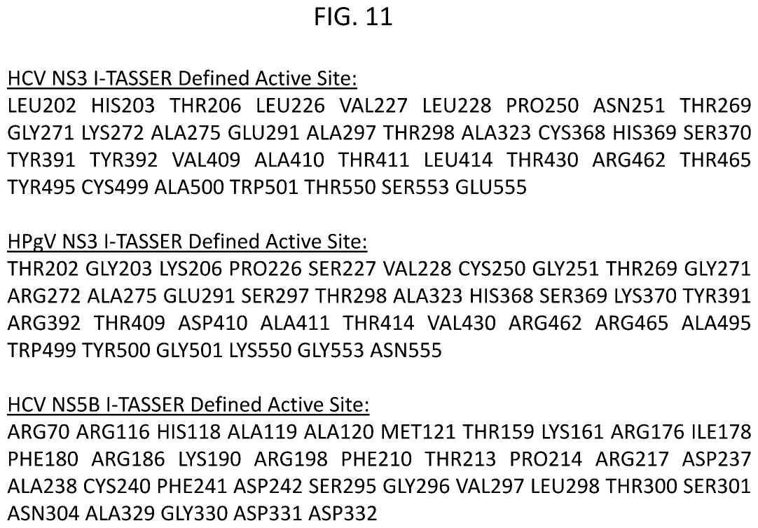

The Hermes graphical user interface (GUI) was used to prepare the protein files for molecular docking via GOLD ³⁶. The proteins were prepared in the GUI by removing any remaining waters and flipping any conformations of charged amino acids as predicted by MolProbity. Molecular docking was completed under default parameters (ChemPLP scoring method). Active sites for HCV-NS3, HPgV-NS3, and HPgV-NS5B were defined by I-TASSER (FIG. 11). (Note: All predicted ligand binding sites were based on pre-existing HCV structures ³⁷⁻⁴⁰). The HCV-NS5B active site was defined by the nesbuvir ligand from a previously published x-ray crystallographic analysis (PDB: 3FQK). Each of the DAAs in FIGS. 12A-12B was docked to their respective protein in silico 10 times. The conformation with the highest fitness score (FS) was retained for further analysis.

### Analysis of Top Drugs

The top five DAAs for each NS protein target chosen for further analysis were those with the lowest difference in HCV docked FS and HPgV docked FS. First, PDBsum was used to generate ligand-protein interactions for the most favorable ligand conformation⁴¹. These drugs were then analyzed based on their pharmacokinetics, drug-likeness, and BBB permeability using SwissADME ⁴². Bioavailability was assessed using the bioavailability radar that provides an “at-a-glance” view of physiochemical properties, such as lipophilicity, size, flexibility, polarity, insolubility, and saturation. Drug-likeness was assessed using Lipinski's Rule of 5 and Brenk Violation parameters⁴². Lipinski's Rule of 5 states that a drug can violate no more than one of the following rules: molar mass less than 500 g/mol, no more than 5 H-bond donors, no more than 10 H-bond acceptors, and a log P less than 5 ⁴³. A Brenk Violation indicates some level of toxicity, chemical reactivity, metabolic instability, or poor pharmacokinetic properties of the drug ⁴². BBB permeability was assessed via the log BB score. The log BB score is a ratio of drug concentration in the brain and blood⁴⁴. Compounds with a log BB>0.3 are considered readily able to cross the BBB while molecules with log BB<−1 are poorly distributed to the brain⁴⁴.

### Molecular Dynamics

Protein-ligand molecular dynamics (MD) simulations were completed for the top HPgV-NS3 (tegobuvir) and HPgV-NS5B (lomibuvir) compounds using the online WebGRO server ⁴⁵⁻⁴⁹. The compounds chosen for MD simulations had the most favorable bioavailability, drug-likeness, and highest likelihood to pass through the BBB. A GROMOS96 43a1 forcefield was used to prepare the protein-ligand complex for MD simulations, and PRODRUG was used to generate ligand topology models ⁵⁰. A Simple Point Charge (SPC) water model was used in a triclinic box type. The system was neutralized with 0.15 M NaCl and the simulation ran for 10 ns under standard parameters (energy minimization step size 5000, temperature 300K, atmospheric pressure).

### REFERENCES

(1) Marano, G.; Franchini, M.; Farina, B.; al., e. The human pegivirus: a new name for an “ancient” virus. can transfusion medicine come up with something new? Acta Virol 2017, 61, 401.
(2) Yu, Y.; Wan, Z.; Wang, J.; Yang, X.; Zhang, C. Review of human pegivirus: Prevalence, transmission, pathogenesis, and clinical implication. Virulence: 2022; Vol. 13, pp 323-340.
(3) Chivero, E. T.; Stapleton, J. T. Tropism of human pegivirus (formerly known as GB virus C/hepatitis G virus) and host immunomodulation: insights into a highly successful viral infection. J Gen Virol: 2015; Vol. 96, pp 1521-1532.
(4) Stapleton, J. T.; Foung, S.; Muerhoff, A. S.; al., e. The GB viruses: a review and proposed classification of GBV-A, GBV-C(HGV), and GBV-D in genus pegivirus within the family Flaviviridae. J Gen Virol: 2011; Vol. 92, pp 233-246.
(5) Moradpour, D.; Penin, F.; Rice, C. M. Replication of hepatitis C virus. Nat Rev Microbiol, 2007; Vol. 5, pp 453-463.
(6) Chivero, E. T.; Bhattarai, N.; McLinden, J. H.; al, e. Human pegivirus (HPgV; formerly known as GBV-C) inhibits IL-12 dependent natural killer cell function. Virology: 2015; Vol. 485, pp 116-127.
(7) Bhattarai, N.; McLinden, J. H.; Xiang, J.; al., e. GB virus C envelope protein E2 inhibits TCR-induced IL-2 production and alters IL-2-signaling pathways. J Immunol., 2012; Vol. 189, pp 2211-2216.
(8) Hebner, C. M.; Han, B.; Brendza, K. M.; Nash, M.; Sulfab, M.; al., e. The HCV Non-Nucleoside Inhibitor Tegobuvir Utilizes a Novel Mechanism of Action to Inhibit NS5B Polymerase Function. PLOS ONE, 2012; Vol. 7, pp e39163.
(9) Handajani, R.; Soetjipto, L.; Suryohudoyo, M. I.; al, e. Prevalence of GB virus C/hepatitis G virus infection among various populations in Surabaya, Indonesia, and identification of novel groups of sequence variants. J Clin Microbiol: 2000; Vol. 32, pp 662-668.
(10) Anggorowati, N.; Y ano, Y.; Subronto, Y. W.; al, e. GB virus C infection in Indonesian HIV-positive patients. Microbiol Immunol, 2013; Vol. 57, pp 298-308.
(11) AbuOdeh, R. O.; Al-Absi, E.; Ali, N. H.; al., e. Detection and phylogenetic analysis of human pegivirus (GBV-C) among blood donors and patients infected with hepatitis B virus (HBV) in Qatar. J Med Virol: 2015; Vol. 87, pp 2074□.
(12) Miao, Z.; Gao, L.; Song, Y.; al, e. Prevalence and clinical impact of human pegivirus-1 infection in HIV-1-infected individuals in Yunnan, China. Viruses: 2017; pp 9-28.
(13) Deuffic-Burban, S.; Delarocque-Astagneau, E.; Abiteboul, D.; al, e. Blood-borne viruses in health care workers: prevention and management. J Clin Virol: 2011; Vol. 52, pp 4-10.
(14) Nordbø, S. A.; Krokstad, S.; Winge, P.; al, e. Prevalence of GB virus C (also called hepatitis G virus) markers in Norwegian blood donors. J Clin Microbiol: 2000; Vol. 38, pp 2584□.
(15) Xiang, J.; Klinzman, D.; McLinden, J.; al, e. Characterization of hepatitis G virus (GB-C virus) particles: evidence for a nucleocapsid and expression of sequences upstream of the E1 protein. J Virol: 1998; Vol. 72, pp 2738□.
(16) Christensen, P. B.; Fisker, N.; Mygind, L. H.; al, e. GB virus C epidemiology in Denmark: different routes of transmission in children and low- and high-risk adults. J Med Virol: 2003; Vol. 70, pp 156 [symbol] .
(17) Balcom, E. F.; Doan, M. A. L.; Branton, W. G.; al, e. Human pegivirus-1 associated leukoencephalitis: clinical and molecular features. Ann Neurol: 2018; Vol. 84, pp 781 [symbol] .
(18) Fridholm, H.; Østergaard Sørensen, L.; Rosenstierne, M. W.; Nielsen, H.; Sellebjerg, F.; Bengård Andersen, A.; Fomsgaard, A. Human pegivirus detected in a patient with severe encephalitis using a metagenomic pan-virus array. Journal of Clinical Virology, 2016; Vol. 77, pp 5-8.
(19) Fama, A.; Xiang, J.; Link, B. K.; al, e. Human pegivirus infection and lymphoma risk and prognosis: a North American study. Br J Haematol: 2018; Vol. 182, pp 644 [symbol] .
(20) Tucker, T. J.; Smuts, H. E.; Eedes, C.; Knobel, G. D.; Eickhaus, P.; Robson, S. C.; al, e. Evidence that the GBV-C/hepatitis G virus is primarily a lymphotropic virus. J Med Virol: 2000; Vol. 1, pp 52-58.
(21) Valyraki, N.; al, e. Human pegivirus identified in severe myelitis and optic neuritis in immunocompromised patients: A pathogenic role for a forgotten virus? Revue neurologique: 2022.
(22) Schibler, M.; Brito, F.; Zanella, M. C.; Zdobnov, E. M.; Laubscher, F.; L'Huillier, A. G.; al., e. Viral sequences detection by highthroughput sequencing in cerebrospinal fluid of individuals with and without central nervous system disease. Genes (Basel): 2019; Vol. 8, pp 625.
(23) Altschul, S. F.; Madden, T. L.; Schäffer, A. A.; Zhang, J.; Zhang, Z.; Miller, W.; Lipman, D. J. Gapped BLAST and PSI-BLAST: a new generation of protein database search programs. Nucleic Acids Research: 1997; Vol. 25, pp 3389-3402.
(24) Waterhouse, A.; Bertoni, M.; Bienert, S.; Studer, G.; Tauriello, G.; Gumienny, R.; Heer, F. T.; de Beer, T. A. P.; Rempfer, C.; Bordoli, L.; et al. SWISS-MODEL: homology modelling of protein structures and complexes. Nucleic Acid Res: 2018; pp W296-W303.
(25) Bienert, S.; Waterhouse, A.; de Beer, T. A. P.; Tauriello, G.; Studer, G.; Bordoli, L.; Schwede, T. The SWISS-MODEL Repository-new features and functionality. Nucleic Acids Res.: 2017; Vol. 45, pp D3313-D3319.
(26) Guex, N.; Peitsch, M. C.; Schwede, T. Automated comparative protein structure modeling with SWISS-MODEL and Swiss-PdbViewer: A historical perspective. Electrophoresis: 2009; Vol. 30, pp S162-S173.
(27) Studer, G.; Tauriello, G.; Bienert, S.; Biasini, M.; Johner, N.; Schwede, T. ProMod3-A versatile homology modelling toolbox. PLOS Comp. Biol.: 2021; Vol. 17, pp e1008667.
(28) Studer, G.; Rempfer, C.; Waterhouse, A. M.; Gumienny, R.; Haas, J.; Schwede, T. Gumienny, R. Bioinformatics: 2020; Vol. 36, pp 1765-1771.
(29) Studer, G.; Biasini, M.; Schwede, T. Assessing the local structural quality of transmembrane protein models using statistical potentials (QMEANBrane). Bioinformatics: 2014; Vol. 30, pp i505-1511.
(30) Benkert, P.; Biasini, M.; Schwede, T. Toward the estimation of the absolute quality of individual protein structure models. Bioinformatics: 2011; Vol. 27, pp 343-350.
(31) Bertoni, M.; Kiefer, F.; Biasini, M.; Bordoli, L.; Schwede, T. Modeling protein quaternary structure of homo- and hetero-oligomers beyond binary interactions by homology. Scientific Reports: 2017.
(32) Mariani, V.; Biasini, M.; Barbato, A.; Schwede, T. IDDT: a local superposition-free score for comparing protein structures and models using distance difference tests. Bioinformatics: 2013; Vol. 29, pp 2722-2728.
(33) Davis, I. W.; Leaver-Fay, V. A.; Chen, V. i. B.; Block, J. N.; Kapral, G. J.; Wang, X.; Murray, L. W.; Arendall III, W. B.; Snoeyink, J.; Richardson, J. S.; et al. MolProbity: all-atom contacts and structure validation for proteins and nucleic acids. Nucleic Acids Research: 2007; Vol. 35, pp W375-W383.
(34) Kim, S.; Chen, J.; Cheng, T.; al., e. PubChem in 2021: new data content and improved web interfaces. Nucleic Acids Res: 2021; Vol. 49, pp D1388-D1395.
(35) O'Boyle, N. M.; Banck, M.; James, C. A.; al, e. Open Babel: An open chemical toolbox. J Cheminform [symbol] ; Vol. 49.
(36) Jones, G. W., P. Glen, R. C. Leach, A. R. Taylor, R. Development and Validation of a Genetic Algorithm for Flexible Docking. J. Mol. Biol.: 1997; Vol. 267, pp 727-748.
(37) Yang, J.; Roy, A.; Zhang, Y. BioLiP: a semi-manually curated database for biologically relevant ligand-protein interactions. Nucleic Acids Research: 2013; Vol. 41, pp D1096-D1103.
(38) Zheng, W.; Zhang, C.; Li, Y.; Pearce, R.; Bell, E. W.; Zhang, Y. Folding non-homology proteins by coupling deep-learning contact maps with I-TASSER assembly simulations. Cell Reports Methods: 2021; Vol. 1, pp 100014.
(39) Zhang, C.; Freddolino, P. L.; Zhang, Y. COFACTOR: improved protein function prediction by combining structure, sequence and protein-protein interaction information. Nucleic Acids Research: 2017; Vol. 45, pp W291-299.
(40) Yang, J.; Zhang, Y. I-TASSER server: new development for protein structure and function predictions. Nucleic Acids Research: 2015; Vol. 43, pp W174-W181.
(41) Laskowski, R. A. PDBsum new things. Nucleic Acids Research: 2009; pp D355-359.
(42) Daina, A.; Michielin, O.; Zoete, V. SwissADME: a free web tool to evaluate pharmacokinetics, drug-likeness and medicinal chemistry friendliness of small molecules. Sci Rep: 2017; Vol. 7, pp 42717.
(43) Lipinski, C. A.; Lombardo, F.; Dominy, B. W.; Feeney, P. J. Experimental and computational approaches to estimate solubility and permeability in drug discovery and development settings. Adv Drug Deliv Rev: 2001; Vol. 46, pp 3-26.
(44) Muehlbacher, M.; Spitzer, G. M.; Liedl, K. R.; Kornhuber, J. Qualitative prediction of blood-brain barrier permeability on a large and refined dataset. J Comput Aided Mol Des: 2011; Vol. 25, pp 1095-1106.
(45) Bekker, H.; Berendsen, H. J. C.; Dijkstra, E. J.; Achterop, S.; van Drunen, R.; van der Spoel, D.; Sijbers, A.; Keegstra, H.; al, e. Gromacs: A parallel computer for molecular dynamics simulations. Physics computing: 1993; pp 252-256.
(46) Abraham, M. J.; Murtola, T.; Schulz, R.; Páll, S.; Smith, J. C.; Hess, B.; Lindahl, E. GROMACS: High performance molecular simulations through multi-level parallelism from laptops to supercomputers. SoftwareX: 2015; Vol. 1-2, pp 19-25.
(47) Lindorff-Larsen, K.; Piana, S.; Palmo, K.; Maragakis, P.; Klepeis, J. L.; Dorr, R. O.; Shaw, D. E. Improved side-chain torsion potentials for the AMBER ff99SB protein force field. PROTEINS: Struct. Funct. Gen: 2010; Vol. 78, pp 1950-1958.
(48) Bjelkmar, P.; Larsson, P.; Cuendet, M. A.; Hess, B.; Lindahl, E. Implementation of the CHARMM force field in GROMACS: Analysis of protein stability effects from correction maps, virtual interaction sites, and water models. J. Chem. Theory Comput.: 2010; Vol. 6, pp 459-466.
(49) Oostenbrink, C.; Villa, A.; Mark, A. E.; Van Gunsteren, W. F. A biomolecular force field based on the free enthalpy of hydration and solvation: The GROMOS force-field parameter sets 53A5 and 53A6. Journal of Computational Chemistry: 2004; Vol. 25, pp 1656-1676.
(50) Schüttelkopf, A. W.; van Aalten, D. M. F. PRODRG: a tool for high-throughput crystallography of protein-ligand complexes. Acta Crystallogr [symbol] ; Vol. D60, pp 1656-1676.
(51) Hang, J.; Yang, Y.; Harris, S.; Leveque, V.; Whittington, H.; Rajyaguru, S.; Ao-leong, G.; McCown, M.; Wong, A.; Giannetti, A.; et al. Slow binding inhibition and mechanism of resistance of non-nucleoside polymerase inhibitors of hepatitis C virus. J Biol Chem.: 2009; Vol. 284, pp 15517-15529.
(52) Laskowski, R. A. Enhancing the functional annotation of PDB structures in PDBsum using key figures extracted from the literature. Bioinformatics: 2007; Vol. 23, pp 1824-1827.
(53) Bhattacharjee, C.; Singh, M.; Das, D.; Chaudhuri, S.; Mukhopadhyay, A. Current therapeutics against HCV. Virusdisease: 2021; Vol. 32, pp 228-243.
(54) Rosenthal. E. S.; Kottilil, S.; Polis. M. A. Sofosbuvir and ledipasvir for HIV/HCV co-infected patients. Expert Opin Pharmacother: 2016; Vol. 17. pp 743-749.
(55) Jin. G.; Lec. J.; Lec. K. Chemical genetics-based development of small molecules targeting hepatitis C virus. Arch. Pharm. Res: 2017; Vol. 40. pp 1021-1036.

What is claimed:

1. A method for diagnosing human pegivirus (HPgV) infection or Parkinson's disease (PD) in a subject, the method comprising: detecting a biomarker associated with HPgV in a sample from the subject.

2. The method of claim 1, wherein the HPgV infection is indicative of Parkinson's disease (PD) or an increased risk for developing PD.

3. The method of claim 1, wherein the sample is a cerebrospinal fluid (CSF) sample, a serum sample, or a blood sample.

4. The method of claim 1, wherein the biomarker is an RNA associated with HPgV, an anti-HPgV antibody, or an HPgV-specific CD8⁺ T cell response.

5. The method of claim 1, wherein the biomarker is detected via reverse transcription polymerase chain reaction (RT-PCR), RNA sequencing, enzyme-linked immunoassay (ELISA), or a CD8⁺ T cell cytotoxicity assay.

6. The method of claim 5, wherein the biomarker is detected via RT-PCR using:
a) one or more primers selected from SEQ ID NOs: 5-9; and/or
b) one or more probes selected from SEQ ID NOs: 10-12.

7. The method of claim 1, wherein the subject is a human.

8. A method for treating HPgV infection or PD in a subject, the method comprising:
administering an anti-hepatitis C virus (HCV) drug to the subject.

9. The method of claim 8, wherein the anti-HCV drug is an NS3-targeting drug or an NS5B-targeting drug.

10. The method of claim 9, wherein the anti-HCV drug is an NS3-targeting drug selected from boceprevir, glecaprevir, grazoprevir, paritaprevir, simeprevir, telaprevir, asunaprevir, danoprevir, narlaprevir, and tegobuvir.

11. The method of claim 9, wherein the anti-HCV drug is an NS5B-targeting drug selected from dasabuvir, sofosbuvir, mercitibane, lomibuvir, 2′-C′-methylguanosine, PSI-7976, Ro-9187, 4′-azidocytidine, and VCH-916.

12. The method of claim 8, wherein the anti-HCV drug is tegobuvir, boceprevir, or lomibuvir.

13. The method claim 8, wherein the anti-HCV drug is modified.

14. The method claim 13, wherein the anti-HCV drug is modified to allow it to cross the blood brain barrier (BBB).

15. The method of claim 8, further comprising detecting a biomarker associated with HPgV in a sample from the subject prior to administering the anti-HCV drug to the subject.

16. The method of claim 15, wherein the sample is a CSF sample, a serum sample, or a blood sample.

17. The method of claim 15, wherein the biomarker is an RNA associated with HPgV, an anti-HPgV antibody, or an HPgV-specific CD8⁺ T cell response.

18. The method of claim 15, wherein the biomarker is detected via RT-PCR, RNA sequencing, ELISA, or a CD8⁺ T cell cytotoxicity assay.

19. The method of claim 18, wherein the biomarker is detected via RT-PCR using:
a) one or more primers selected from SEQ ID NOs: 5-9; and/or
b) one or more probes selected from SEQ ID NOs: 10-12.

20. The method of claim 8, wherein the subject is a human.
